(19) United States
(12) Patent Application Publication
(10) Pub. No.: US 2020/0209259 A1
(43) Pub. Date: Jul. 2, 2020

(54) METHODS TO TARGET PKD1/PKD2 ION CHANNEL COMPLEX

(71) Applicant: Children's Medical Center Corporation, Boston, MA (US)

(72) Inventors: Markus G. Delling, Boston, MA (US); David E. Clapham, Wellesley, MA (US); Julia F. Doerner, Brookline, MA (US)

(73) Assignee: Children's Medical Center Corporation, Boston, MA (US)

(21) Appl. No.: 16/083,479

(22) PCT Filed: Mar. 8, 2017
(86) PCT No.: PCT/US2017/021309

§ 371 Date: Sep. 7, 2018

Related U.S. Application Data
(60) Provisional application No. 62/306,399, filed on Mar. 10, 2016.
(60) Provisional application No. 62/305,160, filed on Mar. 8, 2016.

(51) Int. Cl. G01N 33/68; G01N 33/50; C07K 14/435

(52) U.S. Cl. CPC G01N 33/6872; G01N 33/5008; C07K 2319/01; G01N 2500/10; C07K 2319/20; C07K 14/435

(57) ABSTRACT

Methods for identifying compounds that modulate polycystin-1/polycystin-2 (PKD1/PKD2) ion channel activity or cilium/plasma membrane trafficking are provided. Related reagents and uses of the compounds are also provided.

### RELATED APPLICATIONS

This application claims the benefit under 35 U.S.C. § 119 of U.S. Provisional Application Nos. 62/305,160, filed Mar. 8, 2016, and pending U.S. Application No. 62/306,399, filed Mar. 10, 2016, the entire contents of each of which are incorporated herein by reference.

### BACKGROUND OF INVENTION

Polycystic kidney disease (PKD) is a genetic disease characterized by the growth of numerous fluid-filled cysts in the kidneys. Autosomal dominant polycystic kidney disease (ADPKD) is generally a late-onset multisystem disorder characterized by cysts in the kidneys and other organs, affecting 1 in 800 people. There is currently no treatment for prevention of PKD. Treatments ease the symptoms of ADPKD, and if the kidneys fail, end-stage kidney disease treatments such as dialysis or transplantation are necessary. Mutations are found in the PKD1 and PKD2 genes. PKD1 gene mutations cause ADPKD type 1, and PKD2 gene mutations cause ADPKD type 2.

### SUMMARY OF INVENTION

The invention in some aspects relates to methods for identifying a compound that modulates polycystin-1 (PKD1) and/or polycystin-2 (PKD2) activity or plasma membrane/primary cilium trafficking. The method involves contacting a cell having a plasma membrane PKD1/PKD2 with a test compound, detecting whether PKD1/PKD2 activity is modulated in the presence of the test compound with respect to PKD1/PKD2 activity in the absence of the test compound, and wherein if the PKD1/PKD2 activity is modulated then the test compound is a compound that modulates PKD1/PKD2 activity.

In some embodiments the method of detection comprises a voltage-clamp, patch clamp, x-ray crystallization, electron microscopy, circular dichroism. Fourier transform infra-red spectroscopy, electron spin resonance, nuclear magnetic resonance spectroscopy, flow cytometry, immunodetection fluorescence techniques, surface biotinylation, calcium imaging techniques, or atomic force microscopy.

The test compound in some embodiments comprises a small molecule, peptide, nucleic acid or polysaccharide including, but not limited to antibodies, biologics, an inhibitor of PKD1/PKD2 activity, an activator of PKD1/PKD2 activity, or a trafficking modulator to the plasma membrane or primary cilium.

In some embodiments the PKD1 is a chimera. In other embodiments the PKD2 is a chimera. Optionally the PKD1 and/or PKD2 includes an intracellular or extracellular tag. The tag may be selected from the group consisting of a HA tag, His-tag, GFP, YFP. BirA, mCherry, ires GFP, ices mCherry, FLAG tag, and covalent labeling of fusion proteins using SNAP-, CLIP-, ACP- and MCP-tags.

In some embodiments the N-terminal truncations of PKD1 enhance PKD2 surface trafficking. In other embodiments the C-terminal truncations of PKD1 enhance PKD2 surface trafficking.

In some embodiments PKD1/PKD2 in the plasma membrane comprises a modified PKD1. The modified PKD1 may be, for example, a polycystin-1, modified polycystin-1L1, modified polycystin-1L2, modified polycystin-1L3 with it's extracellular N-terminus replaced by the P2Y12-N-terminus or any other N-terminal domain that enhances surface trafficking of the PKD1/PKD2, complex. In some, embodiments, the N-terminal domain is riot P2Y12.

In other embodiments the modified PKD1/PKD2 complex contains ADPKD disease causing mutations in either PKD1 or PKD2, which either affect plasma membrane trafficking (see FIGS. 14-15) or alter PKD1/PKD2 ion channel function. This approach allows screening for compounds that correct the folding of mutated PKD1 or PKD2 and thus promote plasma membrane/primary cilium trafficking of the PKD1/PKD2 complex.

In other embodiments the PKD1 comprises SEQ ID NO: 3, SEQ ID NO: 4, SEQ ID NO: 5, SEQ ID NO: 6, SEQ ID NO: 7, or SEQ ID NO: 8 and/or the PKD2 comprises SEQ ID NO: 1, SEQ ID NO: 2 or SEQ ID NO: 11.

In other aspects the invention is a cell having a PKD1/PKD2 in a plasma membrane. The PKD1 and/or PKD2 is a chimera. In other embodiments PKD2 contains channel activating or inhibiting mutations. In some embodiments the PKD1 and/or PKD2 includes an intracellular or extracellular tag. The tag may be selected from the group consisting of a HA tag, His-tag, GFP, YFP, BirA, mCherry, ires GFP, ires mCherry, FLAG tag, and covalent labeling of fusion proteins using SNAP-, CLIP-, ACP- and MCP-tags.

In some embodiments the PKD1 in the plasma membrane PKD1/PKD2 comprises a modified PKD1.

In other embodiments the PKD1 comprises SEQ ID NO: 3, SEQ ID NO: 4, SEQ ID NO: 5, SEQ ID NO: 6, SEQ ID NO: 7, or SEQ ID NO: 8 and/or the PKD2 comprises SEQ ID NO: 1, SEQ ID NO: 2 or SEQ ID NO: 11.

The cell in some embodiments is a human embryonic kidney (HEK) cell. In yet other embodiments the cell is an inner medullary collecting duct (IMCD) cell.

The modified PKD1 in some embodiments is a modified polycystin-1L1, modified polycystin-1L2, modified polycystin-1L3 or P2Y12-PKD1.

A chimeric PKD1 comprising a C-terminal PKD1 fragment linked to an N-terminal plasma membrane insertion domain is provided in other aspects of the invention. In some embodiments the plasma membrane insertion domain is P2Y12. In other embodiments the PKD1 includes an intracellular or extracellular tag. In yet other embodiments the tag is selected from the group consisting of a HA tag, His-tag, GFP, mCherry, ires GFP, YFP, BirA, ires mCherry, FLAG tag, and covalent labeling of fusion proteins using SNAP-, CLIP-, ACP- and MCP-tags. The C-terminal PKD1 fragment in some embodiments is SEQ ID NO: 3, SEQ ID NO: 4, SEQ ID NO: 5, SEQ ID NO: 6, SEQ ID NO: 7, or SEQ ID NO: 8. The N-terminal plasma membrane insertion domain in other embodiments is SEQ ID NO: 9. SEQ ID NO: 10.

In other aspects the invention is a nucleic acid encoding a chimeric PKD1 comprising a C-terminal PKD1 fragment linked to an N-terminal plasma membrane insertion domain. In some embodiments, the N-terminal domain is not P2Y12. In some embodiments the nucleic acid encoding the C-terminal PKD1 fragment comprises SEQ ID NO: 3, SEQ ID NO: 4, SEQ ID NO: 5, SEQ ID NO: 6, SEQ ID NO: 7, or SEQ ID NO: 8. In other embodiments the PKD2 comprises SEQ ID NO: 1, SEQ ID NO: 2 or SEQ ID NO: 11. In yet other embodiments the nucleic acid encoding the N-terminal plasma membrane insertion domain is SEQ ID NO: 9 or SEQ ID NO: 10.

A vector, of any of the nucleic acids described herein is provided. In some embodiments the vector comprises an inducible promoter and wherein the inducible promoter consists of an araS promoter, tf55α promoter, araC promoter, or a tetracycline promoter. In other embodiments the vector comprises a dual expression vector (pTRE3G-Bi, Takara Bio) allowing simultaneous expression of PKD1 and PKD2 with similar protein levels (SEQ ID NO: 12 and SEQ ID NO: 13 having SEQ ID NO: 7 and or 8 inserted therein).

In other aspects the invention is a kit having a container housing a first expression vector comprising a nucleic acid encoding a chimeric PKD1 comprising a C-terminal PKD1 fragment linked to an N-terminal plasma membrane insertion domain, a container housing a second expression vector comprising a nucleic acid encoding a PDK2, and instructions for generating a cell line using the first and second expression vectors. In some embodiments the PKD1 and/or the PKD2 is intracellularly or extracellularly tagged. The tag may be selected from the group consisting of a HA tag, His-tag, GFP, YFP, BirA, mCherry, ires GFP, ires mCherry, FLAG tag, and covalent labeling of fusion proteins using SNAP-, CLIP-, ACP- and MCP-tags. In some embodiments the PKD1 comprises SEQ ID NO: 3, SEQ ID NO: 4, SEQ ID NO: 5, SEQ ID NO: 6, SEQ ID NO: 7, or SEQ ID NO: 8. In other embodiments the PKD2 comprises SEQ ID NO: 1, SEQ ID NO: 2 or SEQ ID NO: 11.

A stable inducible cell line expressing PKD1 chimera/PKD2 in the plasma membrane is provided in other aspects of the invention. In some embodiments the cell line consists of inducible and non-inducible HEK cells or CHO cells. In some embodiments, the N-terminal or C-terminal truncations of PKD1 enhance PKD2 surface trafficking. In other embodiments the PKD1 comprises SEQ ID NO: 3, SEQ ID NO: 4, SEQ ID NO: 5, SEQ ID NO: 6, SEQ ID NO: 7, or SEQ ID NO: 8. In yet other embodiments the PKD2 comprises SEQ ID NO: 1, SEQ ID NO: 2 or SEQ ID NO: 11.

In other aspects the invention is a stable inducible cell line expressing PKD1 chimera/PKD2 in the primary cilium. In some embodiments the cell line consists of mIMCD3, hRPE, MDCK or LLC-PK1 cells or other ciliated cell lines. In other embodiments ciliated primary cells for expression of PKD1 chimera/PKD2 are isolated from Arll3B-mCherry-GECO1.2 transgenic mice (FIG. 16) or human tissue. In other embodiments ciliated primary cells also express a calcium sensitive fluorescent protein in primary cilia. In other embodiments the cell line consists of HEK cells or CHO cells. In yet other embodiments the PKD1 comprises SEQ ID NO: 3, SEQ ID NO: 4, SEQ ID NO: 5, SEQ ID NO: 6, SEQ ID NO: 7, or SEQ ID NO: 8. The PKD2 in other embodiments is SEQ ID NO: 1, SEQ ID NO: 2 or SEQ ID NO: 11.

In some embodiments the modified PKD1/PKD2 complex contains ADPKD disease causing imitations in either PKD1 or PKD2 which affects plasma membrane trafficking and/or alters PKD1/PKD2 ion channel function, including gain-of-function mutations in PKD1/PKD2.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

Several methods are disclosed herein of administering to a subject a composition for treatment of a particular condition. It is to be understood that in each such aspect of the invention, the invention specifically includes, also, the composition for use in the treatment of that particular condition, as well as use of the composition for the manufacture of a medicament for the treatment of that particular condition.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1. Polycystin ion channels comprise PKD1 and PKD2 subunits.

FIG. 2. P2Y12-PKD1 chimera reduces the size of PKD1 significantly. SEQ ID NO: 20 is YPYDVPDYA and SEQ ID NO: 21 is QAVDNLTSAPGNTSLCTRDYKITQ.

FIG. 3. Targeting PKD1/P2Y12 chimera to the plasma membrane. The PKD1 chimera is 3.8 kb instead of 12 kb (M[Start] - - - YPYDVPDYA (SEQ ID NO: 20)[HA-tag] - - - QAVDNLTSAPGNTSLCTRDYKITQ (SEQ ID NO: 21) [hP2Y12-NT] - - - ). Four P2Y12-PKD1 chimeras were generated varying in length of PKD1 N-terminus.

FIG. 4. Sequence comparison of human, mouse and rat PKD1 and PKD1L1. SEQ ID NOs: 43-48 from top to bottom, respectively. Positions of P2Y12 fusion to PKD1 N-terminus are indicated with an arrow.

FIG. 5. P2Y12-PKD1-4 localizes to the plasma membrane only with PKD2 present.

FIG. 6. P2Y12-PKD1-3 localizes to the plasma membrane only with PKD2 present.

FIG. 7. P2Y12-PKD1-2 does not localize to the plasma membrane with PKD2 present.

FIG. 8. Surface HA staining is not accomplished beyond position Prot 3a. hPKD1 is shown by SEQ ID NOs: 22-25 from left to right; hPDK1L1 is shown by SEQ ID NOs: 26-29 from left to right; hPDK1L2 is shown by SEQ ID NOs: 30-33 from left to right; hPDK1L3 is shown by SEQ ID NOs: 34-37 from left to right; and hPDK1REJ is shown by SEQ ID NOs: 38-41 from left to right.

FIG. 9. Schematic showing mTRPV3 having an extracellular FLAG insertion allowing quantification of ion channels in the plasma membrane.

FIG. 10. Schematic showing FLAG insertion in extracellular domain of PKD2 using Protter visualization software. The criteria for insertion were 1) different sequences between PKD2 and PKD2-L1 and 2) D/E and K in proximity. DYKDDDDK is SEQ ID NO: 42. A FLAG epitope was inserted at nine different positions and tested for surface immunoreactivity

FIG. 11. Staining of PKD2-FLAG5, the only position that gave a positive FLAG immunostaining.

FIG. 12. Generation of stable cell lines. Reliable co-expression of two proteins is required. Tet-inducible dual promoter was used: Tet-on HEK cell line (Clontech). Tet activator expressing mIMCD3 cell line was generated. Tet-on cell clones with best and worst cilia were selected: IMCD17 and IMCD 28.

FIG. 13. Schematic drawing of assay to screen for plasma membrane and cilia localization of PKD1 and PKD2 proteins simultaneously. A HEK tet-inducible cell line expressing P2Y12-PKD1-4 and PKD2-FLAG generates robust plasma membrane staining for both HA and FLAG. A mIMCD3 tet-inducible cell line expressing P2Y12-PKD1-4 and PKD2-FLAG shows prominent cilia localization of PKD1 and PKD2. Both cell lines were stained live with anti-HA and anti-FLAG antibodies to ensure integrity of the plasma, membrane and thus only immunolabeling of plasma membrane inserted PKD1/PKD2.

FIG. 14. Randomly chosen ADPKD disease causing mutations in PKD1 affect cell surface trafficking of the PKD1/PKD2 complex to various degrees. R3750Q and Q3751X mutations impair surface trafficking of the channel complex

FIG. 15. Randomly chosen ADPKD disease causing mutations in PKD2. All mutations impair surface trafficking of the channel complex.

FIG. 16. Ciliary targeted calcium sensor transgenic mouse model.

FIG. 17. Expression of extracellular domains of PKD1 and PKD1-L1 in yeast (Pichia pastoris).

### DETAILED DESCRIPTION

Autosomal dominant polycystic kidney disease (ADPKD) is a severe disorder which currently has no treatment. Most patients presenting with the disease have a mutation in a PKD1 or PKD2 gene encoding for a membrane spanning polycystin protein present in kidney cells. The function of the polycystin proteins are not well understood and successful therapies targeting the protein have not been developed. It has been discovered, surprisingly, according to the invention, that the polycystin protein complex can be targeted to the plasma membrane and is thus amenable for functional and surface trafficking/protein folding assays and methods described herein. A PKD1 chimera/PKD2 has been developed which is able to be expressed in the plasma membrane. Prior to the instant invention, attempts to develop a functional membrane bound polycystin ion channel complex have not been successful. The ability to target the polycystin ion channel complex to the plasma membrane or primary cilium and to quantify protein levels in plasma or primary cilium membrane using extracellular tags has broad implications for studying the regulation of PKD as well as for identifying modulators of natural channels.

The PKD1 gene provides instructions for making a protein called polycystin-1. The protein consists of a large N-terminus (3000 aa), 11 transmembrane domains and an intracellular C-terminus implicated in the interaction with PKD2. Its positioning in the membrane of kidney cells allows it to interact with other proteins, carbohydrates, and fat molecules (lipids) outside the cell and to receive signals that help the cell respond to its environment. These signals instruct the cell to undergo certain changes, such as maturing to take on specialized functions.

Polycystin-1 is also found in cell structures called primary cilia. Primary cilia are tiny, fingerlike projections that line the small tubes where urine is formed (renal tubules). Researchers believe that primary cilia sense the movement of fluid through these tubules, which appears to help maintain the tubules' size and structure. The interaction of polycystin-1 and polycystin-2 in renal tubules promotes the normal development and function of the kidneys.

The PKD2 gene belongs to a family of genes called TRP (transient receptor potential cation channels) as well as a family of genes called EF-hand domain containing (EF-hand domain containing). The PKD2 gene provides instructions for making a protein called polycystin-2. Polycystin-2 likely functions as a channel spanning the cell membrane of kidney cells. In conjunction with polycystin-1, the channel transports positively charged atoms (ions), including calcium ions, into the cell.

Prior to the instant invention the skilled artisan has not been able to reliably record PKD1/PKD2 currents, because the proteins (full-length) do not traffic to the plasma membrane. As a result a cell based screen for modulators such as agonists or antagonists has not been developed. The methods of the invention involve the discovery of constructs that target the PKD1/PKD2 channel complex to the plasma membrane and thus allow functional characterization and screening for activators and inhibitors of the ion channel complex. In particular the invention involves a PKD1 chimera/PKD2 that is modified to achieve membrane expression. For instance, a chimera of an N-terminal protein fused to a truncated form of PKD1 can enhance the cell surface trafficking. In some embodiments, a chimera of the invention is a N-terminal protein-PKD1 fragment.

An N-terminal protein, as used herein is a signal peptide that enhances trafficking to the plasma membrane. In some embodiments, the peptide is signal peptide. Preferably the N-terminal protein is an N-terminal region of a GPCR. GPCR's are well known in the art. In some embodiments the N terminal protein is an N-terminus of P2Y12. other embodiments the N-terminus is a 20-30 amino acid N-terminus of a cell surface protein that promotes cell surface delivery.

A PKD1 fragment is a functionally active fragment of PKD1. In some embodiments the PKD1 fragment is a C-terminal fragment of PKD1. It may be between 500 and 1,500 amino acids in length in some embodiments. Thus, in some embodiments, the PKD1 is a chimera, wherein N-terminal truncations of PKD1 enhance PKD2 surface trafficking. In some embodiments, the PKD1 is a chimera, wherein C-terminal truncations of PKD1 enhance PKD2 surface trafficking. In another embodiment the functionally active fragment of PKD1 is the entire extracellular domain of PKD1 or PKD1-L1 or PKD1-L2 or PKD1-L3 or fragments thereof expressed and purified from heterologous expression systems such as Pichia pastoris, insect cells or mammalian cell lines such as HEK or CHO cells. It may be between 200 and 3000 amino acids in length.

Aspects of the invention relate to in vitro and/or in vivo assays for identifying compounds that reduce the negative effects of defects in PKD1/PKD2. In some embodiments, a candidate compound is identified in an assay as modulating ion transport function of PKD1/PKD2. Aspects of the invention may be implemented in any suitable assay format, including, for example, a high throughput assay format. For example, a high throughput screen (HTS) format of more than about 10,000, more than 100,000 (e.g., >110,000) compounds may be used to identify compounds that modulate PKD1/PKD2 activity. In some embodiments, a first screen (e.g., high throughput screens) may be used to identify one or more candidate compounds that have at least a threshold effect on PKD1/PKD2 activity. In some embodiments, a threshold effect may be an increase or decrease of PKD1/PKD2 activity of at least 5%, at least 10%, 10-20%, 20-30%, 30-40%, 40-50%, 50-60%, 60-70%, 70-80% 80-90%, 90-95% 95%-100% relative to a control level of PKD1/PKD2 activity in the absence of a compound or in the presence of a compound that is known to have little or no effect on PKD1/PKD2 activity (negative control) or to have a modulatory effect (positive control).

In some embodiments, a candidate compound that is identified in a first screen may be evaluated in a second screen to confirm that it is useful as a therapeutic agent. Thus, the compound may be further evaluated in vitro or in vivo to determine whether it selectively promotes or inhibits physiological function in the cell. Accordingly, a cell-based PKD1/PKD2 assay in the presence or absence of a test compound may be used in conjunction or independently of an in vitro assay to evaluate further physiological effects of candidate test compounds. It should be appreciated that in some embodiments downstream readouts associated with PKD1/PKD2 activity also may be used to evaluate the effects of one or more test compounds.

In some embodiments, the test compound comprises a small molecule, peptide, nucleic acid or polysaccharide including, but not limited to antibodies and biologics.

In some embodiments, the test compound comprises an inhibitor of PKD1/PKD2 activity. In other embodiments, the test compound comprises an activator of PKD1/PKD2 activity. In some embodiments, the test compound comprises a trafficking modulator. In some embodiments, the test compound comprises a trafficking modulator to the plasma membrane or primary cilium that corrects misfolding of PKD1 or PKD2 due to point mutations in the proteins resulting in reduced plasma membrane trafficking of the protein complex. In some embodiments the test compound or candidate compound is a modulator of PKD1/PKD2 activity. A modulator of PKD1/PKD2 activity is an agonist, an antagonist, an inverse agonist, a positive allosteric regulator or a negative allosteric regulator of PKD1/PKD2 activity. An “agonist” is a molecule capable, on its own, of increasing the activity of PKD1/PKD2. An “antagonist” is a molecule capable of inhibiting the activating effect of an agonist. An “inverse agonist” is a molecule capable of inhibiting the constitutive activity of PKD1/PKD2, i.e. the activity measurable in the absence of any agonist, when such an activity is effectively measurable. An inverse agonist is also capable of inhibiting the effect of an agonist. It is therefore also an antagonist. A “positive allosteric regulator” is a molecule capable of facilitating the action of an agonist. A “negative allosteric regulator” is a molecule capable of decreasing the effect of an agonist.

It should be appreciated that any conditions associated with aberrant PKD1/PKD2 activity may be treated with the compounds identified according to the screening methods of the invention The compounds identified in the screening assays described herein are useful as therapeutic, diagnostic and/or research reagents. Mutations in polycystin 1 and 2 manifest in severe ciliopathies. Thus, compounds that modulate PKD1/PKD2 activity may be useful as therapeutic agents. For instance the active compounds may be therapeutic agents in the treatment of kidney disease such as Autosomal dominant polycystic kidney disease (ADPKD), cyst formation, disorders involving cilia such as meduloblastoma, glioblastoma, and basal cell carcinoma, and vascular disease such as cardiac vascular disease.

ADPKD is generally a late-onset multisystem disorder characterized by: bilateral renal cysts; cysts in other organs including the liver, seminal vesicles, pancreas, and arachnoid membrane; vascular abnormalities including intracranial aneurysms, dilatation of the aortic root, and dissection of the thoracic aorta; mitral valve prolapse; and abdominal wall hernias. Renal manifestations include hypertension, renal pain, and renal insufficiency. Approximately 50% of individuals with ADPKD have end-stage renal disease (ESRD) by age 60 years. About 95% of individuals with ADPKD have an affected parent; at least 10% of families can be traced to de novo imitation. Polycystic kidney disease 1 (autosomal dominant) is also known as Lov-1, PBP, Pc-1, PC1, TRPP1, and PKD1. Polycystic kidney disease 2 (autosomal dominant) is also known as APKD2, PC2, PKD4, Pc-2, TRPP2, and PKD2.

The prevalence of liver cysts, the most common extrarenal manifestation of ADPKD, increases with age and may have been underestimated by ultrasound studies. The prevalence of intracranial aneurysms is higher in those with a positive family history of aneurysms or subarachnoid hemorrhage (22%) than in those without such a family history (6%). Mitral valve prolapse, the most common valvular abnormality, occurs in up to 25% of affected individuals. Substantial variability in severity of renal disease and other extrarenal manifestations occurs even within the same family. The diagnosis of ADPKD is established primarily by imaging studies of the kidneys. In approximately 85% of individuals with ADPKD, pathogenic variants in PKD1 are causative; in approximately 15% of individuals, pathogenic variants in PKD2 are causative.

Therapeutic interventions aimed at slowing the progression of ESRD in ADPKD include control of hypertension and hyperlipidemia, dietary protein restriction, control of acidosis, and prevention of hyperphosphatemia. Most individuals with polycystic liver disease have no symptoms and require no treatment. The mainstay of therapy for ruptured or symptomatic intracranial aneurysm is surgical clipping of the ruptured aneurysm at its neck; however, for some individuals, endovascular treatment with detachable platinum coils may be indicated. Thoracic aortic replacement is indicated when the aortic root diameter exceeds established size (Harris P C, Torres V E. Polycystic Kidney Disease, Autosomal Dominant. 2002 Jan. 10 [Updated 2015 Jun. 11]. In: Pagon R A, Adam M P. Ardinger H H, et al., editors. GENEREVIEWS® [Internet]. Seattle (Wash.): University of Washington, Seattle; 1993-2016).

In some embodiments, methods of detection comprise a voltage-clamp, patch clamp, x-ray crystallization, electron microscopy, circular dichroism. Fourier transform infra-red spectroscopy, electron spin resonance, nuclear magnetic resonance spectroscopy, flow cytometry, immunodetection fluorescence techniques, surface biotinylation, calcium imaging techniques, or atomic force microscopy. These methods assist in identifying a compound that modulates polycystin-1/polycystin-2 (PKD1/PKD2) activity, comprising contacting a cell having a plasma membrane PKD1/PKD2 with a test compound, detecting whether PKD1/PKD2 activity is modulated in the presence of the test compound with respect to PKD1/PKD2 activity in the absence of the test compound, and wherein if the PKD1/PKD2 activity is modulated then the test compound is a compound that modulates PKD1/PKD2 activity.

In some embodiments, the PKD1 is a chimera, wherein the PKD1 includes an intracellular or extracellular tag. In some embodiments, the PKD2 is a chimera, wherein the PKD2 includes an intracellular or extracellular tag.

In some embodiments, the tag can be, but is not limited to a HA tag, His-tag, GFP, YFP, BirA, mCherry, ires GFP, ires mCherry, FLAG tag, and covalent labeling of fusion proteins using SNAP-, CLIP-, ACP- and MCP-tags.

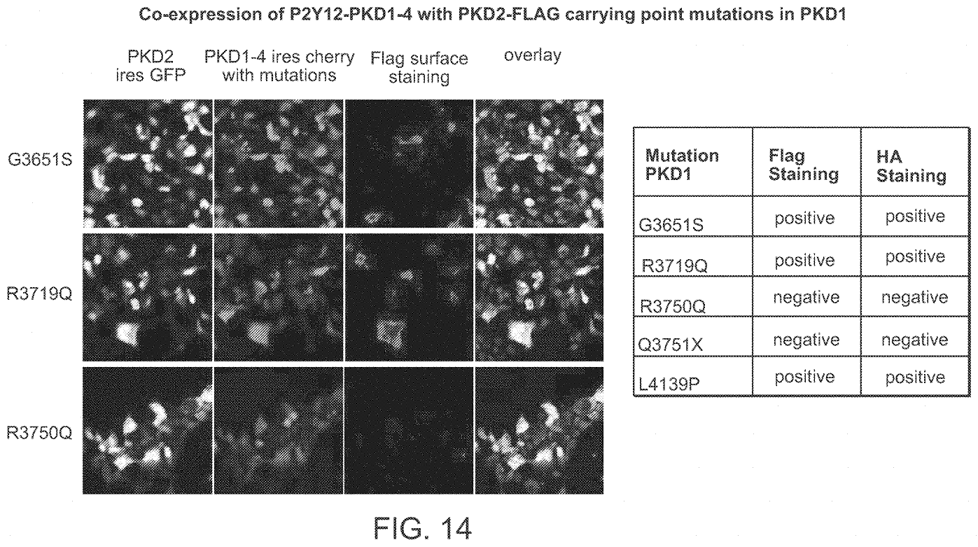

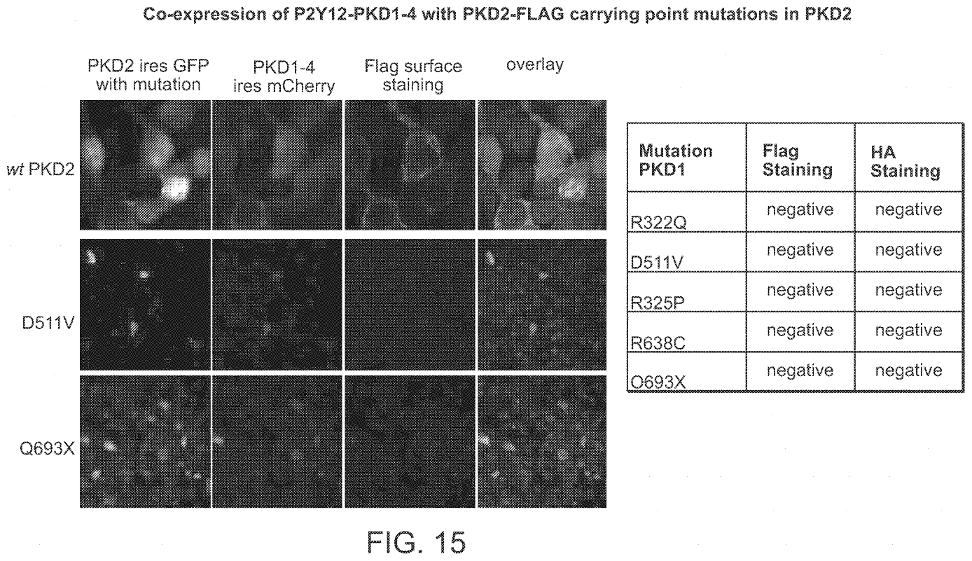

In other embodiments the modified PKD1/PKD2 complex contains ADPKD disease causing mutations in either PKD1 or PKD2, which either affect plasma membrane trafficking (see FIGS. 14-15) or alter PKD1/PKD2 ion channel function. This approach allows screening for compounds that correct the folding of mutated PKD1 or PKD2 and thus promote plasma membrane/primary cilium trafficking of the PKD1/PKD2 complex.

A nucleic acid or polypeptide sequence that is “derived from” a designated sequence refers to a sequence that corresponds to a region of the designated sequence. For nucleic acid sequences, this encompasses sequences that are homologous or complementary to the sequence, as well as “sequence-conservative variants” and “function-conservative variants.” For polypeptide sequences, this encompasses “function-conservative variants.” Sequence-conservative variants are those in which a change of one or more nucleotides in a given codon position results in no alteration in the amino acid encoded at that position. Function-conservative variants are those in which a given amino acid residue in a polypeptide has been changed without altering the overall conformation and function of the native polypeptide, including, but not limited to, replacement of an amino acid with one having similar physico-chemical properties (such as, for example, acidic, basic, hydrophobic, and the like). “Function-conservative” variants also include any polypeptides that have the ability to elicit antibodies specific to a designated polypeptide.

Conservative substitutions typically include the substitution of one amino acid for another with similar characteristics, e.g., substitutions within the following groups: valine, glycine; glycine, alanine: valine, isoleucine, leucine; aspartic acid, glutamic acid; asparagine, glutamine; serine, threonine; lysine, arginine; and phenylalanine, tyrosine. Other conservative substitutions are known in the art. The invention encompasses sequence-conservative variants and function-conservative variants of these sequences. The nucleic acids may be DNA, RNA, DNA/RNA duplexes, protein-nucleic acid (PNA), or derivatives thereof.

In some embodiments, the PKD I in the plasma membrane PKD1/PKD2 comprises a modified PKD1. In some embodiments, the modified PKD1 is a P2Y12-PKD1, P2Y12-1L1, P2Y12-1L2, or P2Y12-1L3. In some embodiments, the PKD1 comprises SEQ ID NO: 3, SEQ ID NO: 4, SEQ ID NO: 5, SEQ ID NO: 6, SEQ ID NO: 7, SEQ ID NO: 8 or sequence-conservative or function-conservative variants thereof. In some embodiments, the PKD2 comprises SEQ ID NO: 1, SEQ ID NO: 2, SEQ ID NO: 11 or sequence-conservative or function-conservative variants thereof.

In some embodiments, the invention consists of a cell having a PKD1/PKD2 in a plasma membrane, wherein the PKD1 is a chimera and/or the PKD2 is a chimera. In some embodiments, the PKD1 includes a tag at the PKD2 includes a tag. In some embodiments, the tag can be, but is not limited to a HA tag, His-tag, GFP, YFP, BirA, mCherry, ires GFP, ires mCherry, FLAG tag, and covalent labeling of fusion proteins using SNAP-, CLIP-, ACP- and MCP-tags. In some embodiments, the PKD1 in the plasma membrane PKD1/PKD2 comprises a modified PKD1. For example, the PKD1 can comprise SEQ ID NO: 3, SEQ ID NO: 4, SEQ ID NO: 5, SEQ ID NO: 6, SEQ ID NO:7, SEQ ID NO: 8 or sequence-conservative or function-conservative variants thereof. The PKD2 can comprise SEQ ID NO: 1, SEQ ID NO: 2, SEQ ID NO: 11 or sequence-conservative or function-conservative variants thereof.

In some embodiments, the cell is a human embryonic kidney (HEK) cell. In other embodiments, the cell is an inner medullary collecting duct (IMCD) cell. In some embodiments, the modified PKD1 is a modified polycystin-1L1, modified polycystin-1L2, modified polycystin-1L3 or P2Y12-PKD1. In some embodiments, the N-terminus of the modified PKD1 does not contain P2Y12.

In some embodiments, the invention includes an inducible cell line expressing PKD1 chimera/PKD2 in the plasma membrane. In some embodiments, the cell line consists of HEK cells or CHO cells. In some embodiments the cell line used for inducible PKD1/PKD2 expression contains a primary cilium such as mIMCD3, hRPE, MDCK or LLC-PK1 cells. This allows quantification of protein trafficking to both plasma membrane and primary cilium. In some embodiments, the PKD1 comprises SEQ ID NO: 3, SEQ ID NO: 4, SEQ ID NO: 5, SEQ ID NO: 6, SEQ ID NO: 7, SEQ ID NO: 8 or sequence-conservative or function-conservative variants thereof. In other embodiments, the PKD2 comprises SEQ ID NO: 1, SEQ ID NO: 2, SEQ ID NO: 11 or sequence-conservative or function-conservative variants thereof.

In some embodiments, the invention includes a stable inducible cell line expressing PKD1 chimera/PKD2 in the primary cilium. In some embodiments, the cell line consists of IMCD3 cells or other ciliated cell lines or ciliated primary cells. In some embodiments, the invention cell line consists of HEK cells or CHO cells. In some embodiments, the PKD1 comprises SEQ ID NO: 3, SEQ ID NO: 4, SEQ ID NO: 5, SEQ ID NO: 6, SEQ ID NO: 7, SEQ ID NO: 8 or sequence-conservative or function-conservative variants thereof. In other embodiments, the PKD2 comprises SEQ ID NO: 1, SEQ ID NO: 2, SEQ ID NO: 11 or sequence-conservative or function-conservative variants thereof.

In some embodiments, the invention includes a chimeric PKD1 comprising a C-terminal PKD1 fragment linked to an N-terminal plasma membrane insertion domain. In other embodiments, the plasma membrane insertion domain is P2Y12. The PKD1 can, in some embodiments, include an extracellular tag. In some embodiments, the C-terminal PKD1 fragment comprises SEQ ID NO: 3, SEQ ID NO: 4, SEQ ID NO: 5, SEQ ID NO: 6, SEQ ID NO: 7, SEQ ID NO: 8 or sequence-conservative or function-conservative variants thereof. In other embodiments, the N-terminal plasma membrane insertion domain comprises SEQ ID NO: 9, SEQ ID NO: 10 or sequence-conservative or function-conservative variants thereof.

In some embodiments, the invention includes a nucleic acid encoding a chimeric PKD1 comprising a C-terminal PKD1 fragment linked to an N-terminal plasma membrane insertion domain. In some embodiments, the C-terminal PKD1 fragment comprises SEQ ID NO: 3, SEQ ID NO: 4, SEQ ID NO: 5, SEQ ID NO: 6, SEQ ID NO: 7, SEQ ID NO: 8 or sequence-conservative or function-conservative variants thereof. In other embodiments, the N-terminal plasma membrane insertion domain comprises SEQ ID NO: 1, SEQ ID NO: 2, SEQ ID NO: 10 or sequence-conservative or function-conservative variants thereof. In some embodiments, the nucleic acid encoding the N-terminal plasma membrane insertion domain is SEQ ID NO: 9, SEQ ID NO: 10 or sequence-conservative or function-conservative variants thereof.

In some embodiments, the sequences are of human origin. In other embodiments, the sequences can be of xenarthra, Chiroptera, camivora, cetacea, dermoptera, macroscelidea, proboscidea, lagomorphs, artiodactyla, perissodactyls, hyracoidea, insectivora, marsupialia, monotremata, pholidota, primates, rodentia, pinnipedia, sirenia, or scandentia origins.

In some embodiments, engineered constructs and/or engineered nucleic acids are included within a “vector”. A vector is a nucleic acid (e.g., DNA) used as a vehicle to artificially carry genetic material (e.g., an engineered nucleic acid) into another cell where, for example, it can be replicated and/or expressed.

Biological vectors include, but are not limited to, plasmids, phagemids, viruses, other vehicles derived from viral or bacterial sources that have been manipulated by the insertion or incorporation of the nucleic acid sequences of the invention, and free nucleic acid fragments which can be linked to the nucleic acid sequences of the invention. A non-limiting example of a vector is a plasmid. Plasmids are double-stranded generally circular DNA sequences that are capable of automatically replicating in a host cell. Plasmd vectors typically contain an origin of replication that allows for semi-independent replication of the plasmid in the host and also the transgene insert. Plasmids may have more features, including, for example, a “multiple cloning site,” which includes nucleotide overhangs for insertion of a nucleic acid insert, and multiple restriction enzyme consensus sites to either side of the insert.

Viral vectors are a preferred type of biological vector and include, but are not limited to, nucleic acid sequences from the following viruses: retroviruses, such as Moloney murine leukemia virus; Harvey murine sarcoma virus; murine mammary tumor virus; Rous sarcoma virus; adenovirus; adeno-associated virus; SV40-type viruses; polyoma viruses; poxviruses; Epstein-Barr viruses; papilloma viruses; herpes virus; vaccinia virus; and polio virus. One can readily employ other vectors not named but known in the art.

In some embodiments, the invention consists of a vector comprising a nucleic acid encoding a chimeric PKD1 comprising a C-terminal PKD1 fragment linked to an N-terminal plasma membrane insertion domain. In some embodiments, the vector comprises SEQ ID NO: 12, SEQ ID NO: 13 or sequence-conservative or function-conservative variants thereof.

Engineered constructs of the present disclosure comprise, in some embodiments, promoters operably linked to a nucleotide sequence (e.g., encoding a protein of interest). A “promoter” is a control region of a nucleic acid at which initiation and rate of transcription of the remainder of a nucleic acid are controlled. A promoter may also contain sub-regions at which regulatory proteins and molecules may bind, such as RNA polymerase and other transcription factors. Promoters may be constitutive, inducible, activatable, repressible, tissue-specific or any combination thereof.

A promoter drives expression or drives transcription of the nucleic acid sequence that it regulates. A promoter is considered to be “operably linked” when it is in a correct functional location and orientation in relation to the nucleotide sequence it regulates to control (“drive”) transcriptional initiation and/or expression of that sequence.

Promoters of an engineered nucleic acid construct may be “inducible promoters,” which refer to promoters that are characterized by regulating (e.g., initiating or activating) transcriptional activity when in the presence of, influenced by or contacted by an inducer signal. An inducer signal may be endogenous or a normally exogenous condition (e.g., light), compound (e.g., chemical or non-chemical compound) or protein that contacts an inducible promoter in such a way as to be active in regulating transcriptional activity from the inducible promoter. Thus, a “signal that regulates transcription” of a nucleic acid refers to an inducer signal that acts on an inducible promoter. A signal that regulates transcription may activate or inactivate transcription, depending on the regulatory system used. Activation of transcription may involve directly acting on a promoter to drive transcription or indirectly acting on a promoter by inactivating a repressor that is preventing the promoter from driving transcription. Conversely, deactivation of transcription may involve directly acting on a promoter to prevent transcription or indirectly acting on a promoter by activating a repressor that then acts on the promoter.

The administration or removal of an inducer signal results in a switch between activation and inactivation of the transcription of the operably linked nucleic acid sequence. Thus, the active state of a promoter operably linked to a nucleic acid sequence refers to the state when the promoter is actively regulating transcription of the nucleic acid sequence (i.e., the linked nucleic acid sequence is expressed). Conversely, the inactive state of a promoter operably linked to a nucleic acid sequence refers to the state when the promoter is not actively regulating transcription of the nucleic acid sequence (i.e., the linked nucleic acid sequence is not expressed).

An inducible promoter of the present disclosure may be induced by (or repressed by) one or more physiological condition(s), such as changes in light, pH, temperature, radiation, osmotic pressure, saline gradients, cell surface binding, and the concentration of one or more extrinsic or intrinsic inducing agent(s). An extrinsic inducer signal or inducing agent may comprise, without limitation, amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones or combinations thereof.

Inducible promoters of the present disclosure include any inducible promoter described herein or known to one of ordinary skill in the art. In some embodiments, the inducible promoter consists of an araS promoter, tf55α promoter, araC promoter, or a tetracycline promoter. Other examples of inducible promoters include, without limitation, chemically/biochemically-regulated and physically-regulated promoters such as alcohol-regulated promoters, tetracycline-regulated promoters (e.g., anhydrotetracyclinc (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH), temperature/heat-inducible promoters (e.g., heat shock promoters), and light-regulated promoters (e.g., light responsive promoters from plant cells).

In some embodiments, inducible promoters of the present disclosure function in prokaryotic cells (e.g., bacterial cells). Examples of inducible promoters for use prokaryotic cells include, without limitation, bacteriophage promoters (e.g. Pls1con, T3, T7, SP6, PL) and bacterial promoters (e.g., Pbad, PmgrB, Ptrc2, Plac/ara, Ptac, Pm), or hybrids thereof (e.g. PLlacO, PLtetO). In some embodiments, inducible promoters of the present disclosure function in eukaryotic cells (e.g., mammalian cells). Examples of inducible promoters for use eukaryotic cells include, without limitation, chemically-regulated promoters (e.g., alcohol-regulated promoters, tetracycline-regulated promoters, steroid-regulated promoters, metal-regulated promoters, and pathogenesis-related (PR) promoters) and physically-regulated promoters (e.g., temperature-regulated promoters and light-regulated promoters).

In some embodiments, the invention includes a kit comprising a container housing a first expression vector comprising a nucleic acid encoding a chimeric PKD1 comprising a C-terminal PKD1 fragment linked to an N-terminal plasma membrane insertion domain, a container housing a second expression vector comprising a nucleic acid encoding a PKD2, and instructions for generating a cell line using the first and second expression vectors. In some embodiments PKD1 and PKD2 are dually expressed from the same expression vector. In some embodiments, the PKD1 is tagged. In other embodiments, the PKD2 is tagged. In some embodiments, the tag is selected from the group consisting of a HA tag, His-tag, GFP, YFP; BirA, mCherry, ires GFP, ires mCherry, FLAG tag, and covalent labeling of fusion proteins using SNAP-, CLIP-, ACP- and MCP-tags. In some embodiments, the PKD1 comprises SEQ ID NO: 3, SEQ ID NO: 4, SEQ ID NO: 5, SEQ ID NO: 6, SEQ ID NO: 7, SEQ ID NO: 8 or sequence-conservative or function-conservative variants thereof. In other embodiments, the PKD2 comprises SEQ ID NO: 1, SEQ ID NO: 2, SEQ ID NO: 11 or sequence-conservative or function-conservative variants thereof. That is, the kit can include a description of use of the compositions as discussed herein. Instructions also may be provided for producing cells and/or screening cells by any suitable technique.

The kits described herein may also contain one or more containers, which may contain the composition and other ingredients as previously described. The kits also may contain instructions for mixing, diluting, and/or administrating the compositions of the invention in some cases.

The present invention is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1

### Autosomal Dominant Polycystic Kidney Disease

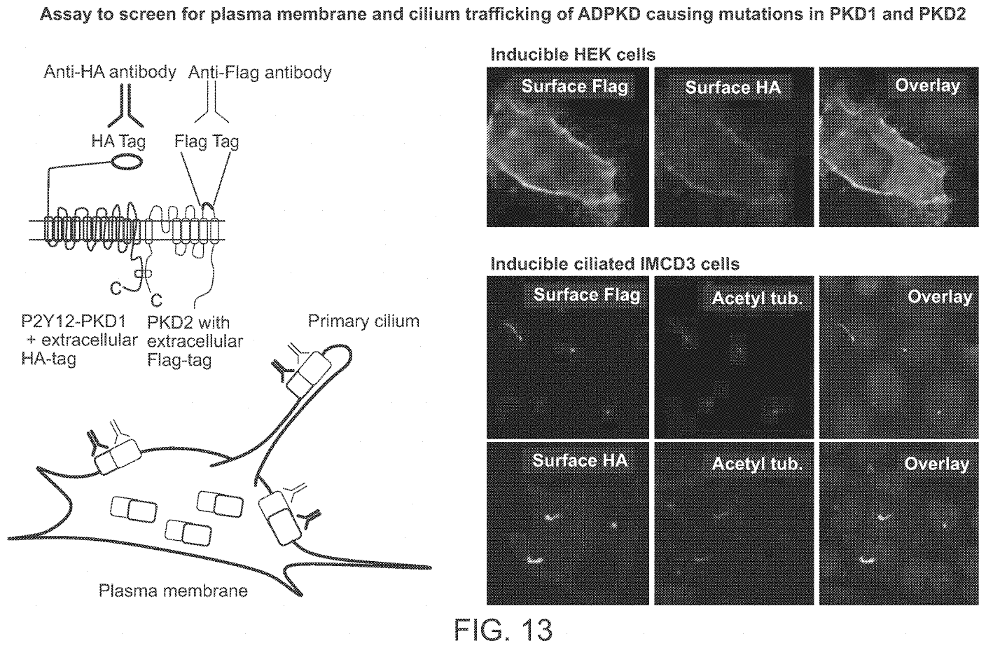

ADPKD mutations occur in a putative receptor ion channel complex. PKD2 is a member of the transient channel family (TRP channels). PKD1 and PKD2 localize to primary cilia (FIG. 13).

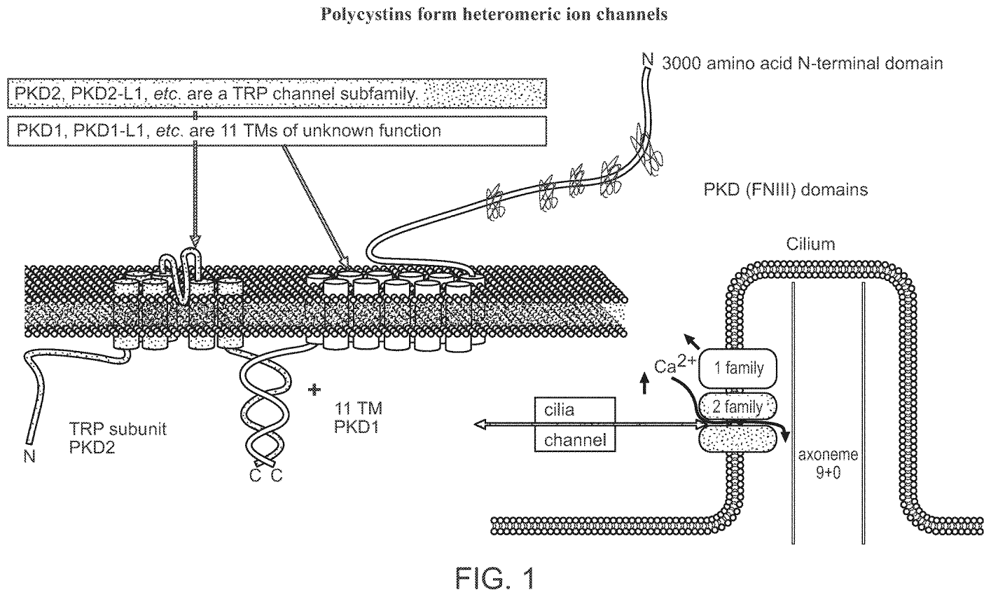

PKD1 and PKD1-L1 have 11 transmembranes of unknown function. PKD2 and PKD2-L1 are in the TRP channel subfamily. Polycystins form heteromeric complexes (FIG. 1).

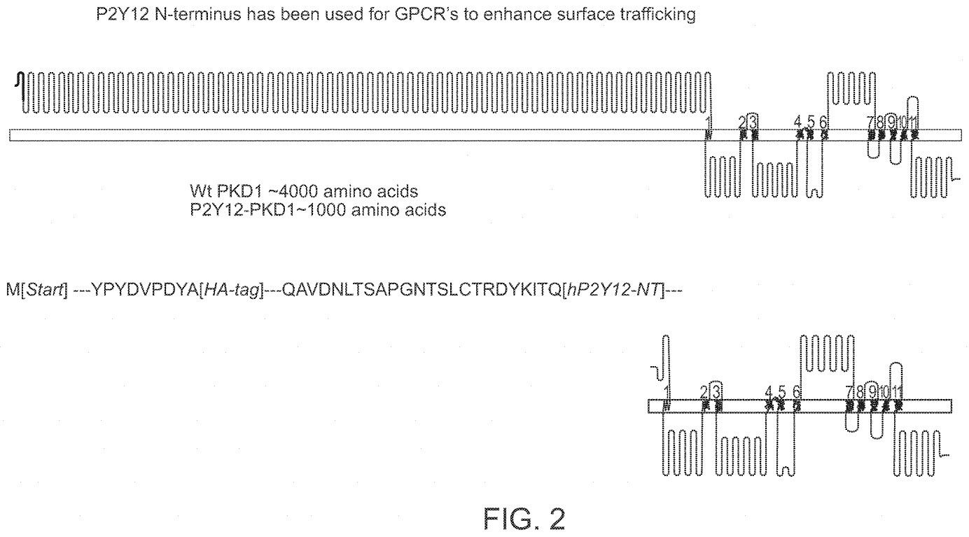

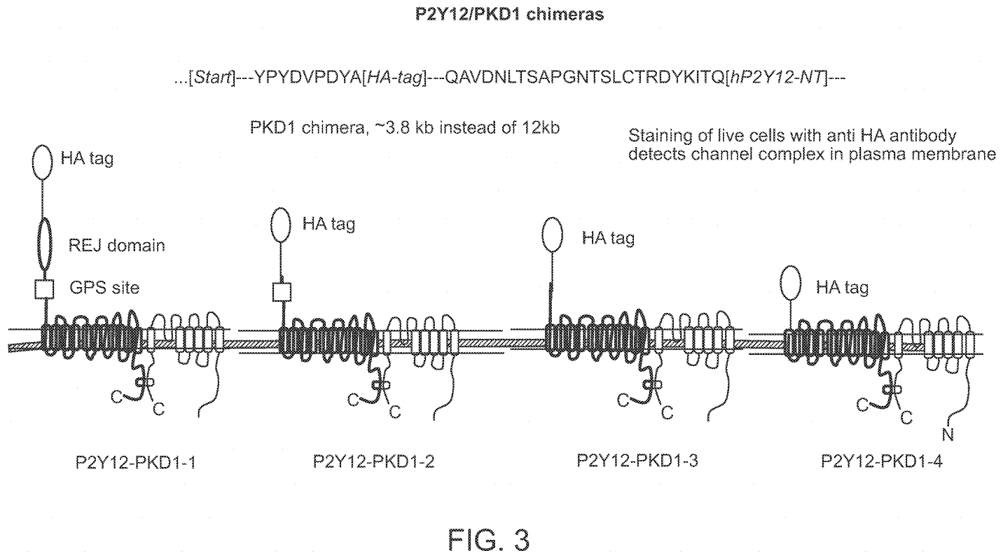

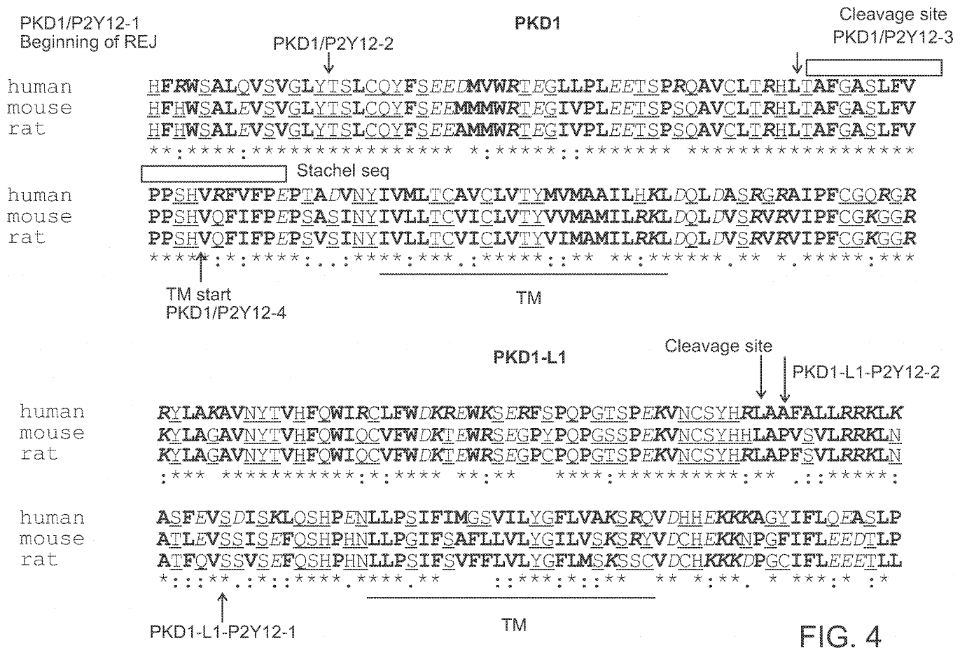

P2Y12-PKD1 chimera reduces the size of PKD1 significantly (FIG. 2). P2Y12 N-terminus has been used for GPCR's to enhance surface trafficking. SEQ ID NO: 20 is YPYDVPDYA and SEQ ID NO: 21 is QAVDNLTSAPGNTSLCTRDYKITQ. The wild-type PKD1 consists of approximately 4000 amino acids, whereas the P2Y12-PKD1 consists of approximately 1000 amino acids. Four PKD1 chimeras were generated and tested (FIG. 3, 4). FIG. 4 shows sequence comparisons of human, mouse and rat PKD1 and PKD1L1 with position of P2Y12 fusion to PKD1 indicated by an arrow, SEQ ID NOs: 43-48 from top to bottom, respectively.

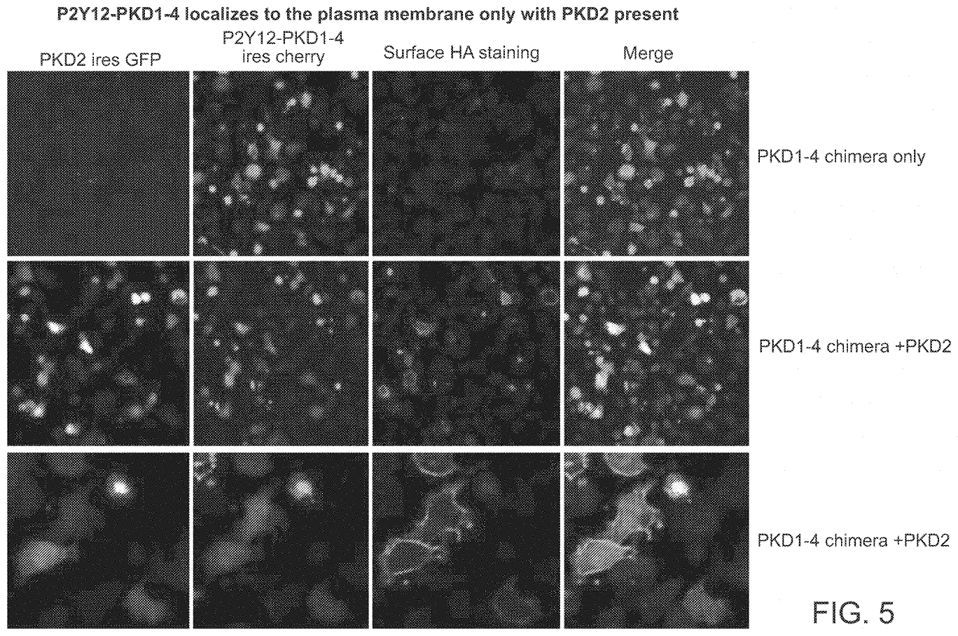

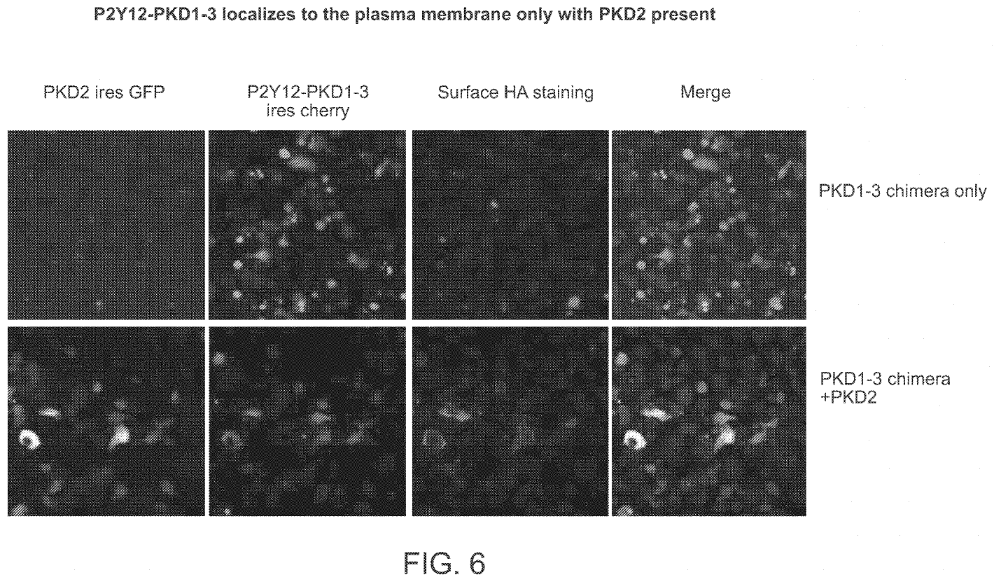

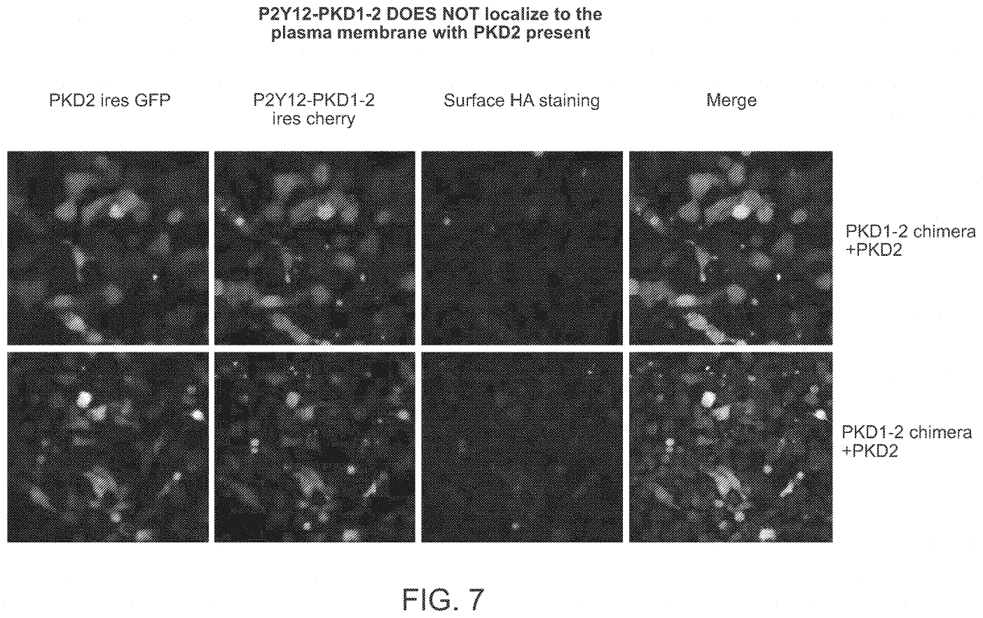

Next, an experiment was set-up to identify if a PKD1/P2Y12 chimera could be targeted to the plasma membrane. Various chimeras were tested. The PKD1 chimera is 3.8 kb instead of 12 kb (M[Start] - - - YPYDVPDYA (SEQ ID NO: 20)[HA-tag] - - - QAVDNLTSAPGNTSLCTRDYKITQ (SEQ ID NO: 21)[hP2Y12-NT] - - - ). Staining of live cells with anti-HA antibody detects the PKD1 chimera in plasma membrane. P2Y12-localized to the plasma membrane only with PKD2 present. (FIG. 5). P2Y12-PKD1-3 localized to the plasma membrane only with PKD2 present (FIG. 6). P2Y12-PKD1-2 did not localize to the plasma membrane with PKD2 present (FIG. 7).

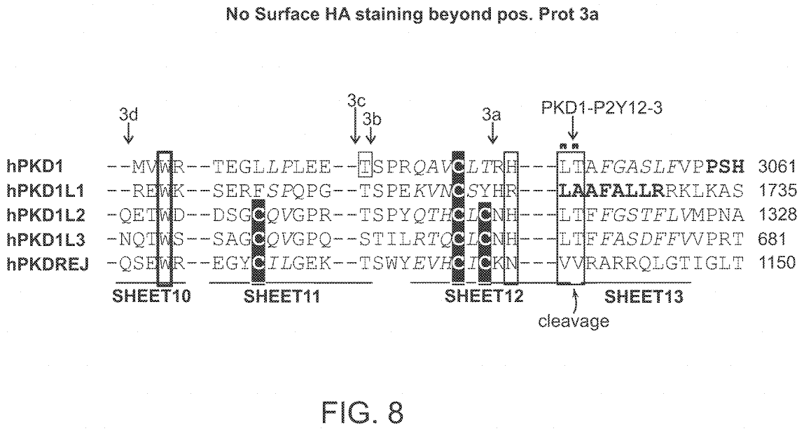

FIG. 8 shows that surface HA staining is not accomplished beyond N-terminal fusion of P2Y12 to PKD1 at position Prot 3a.

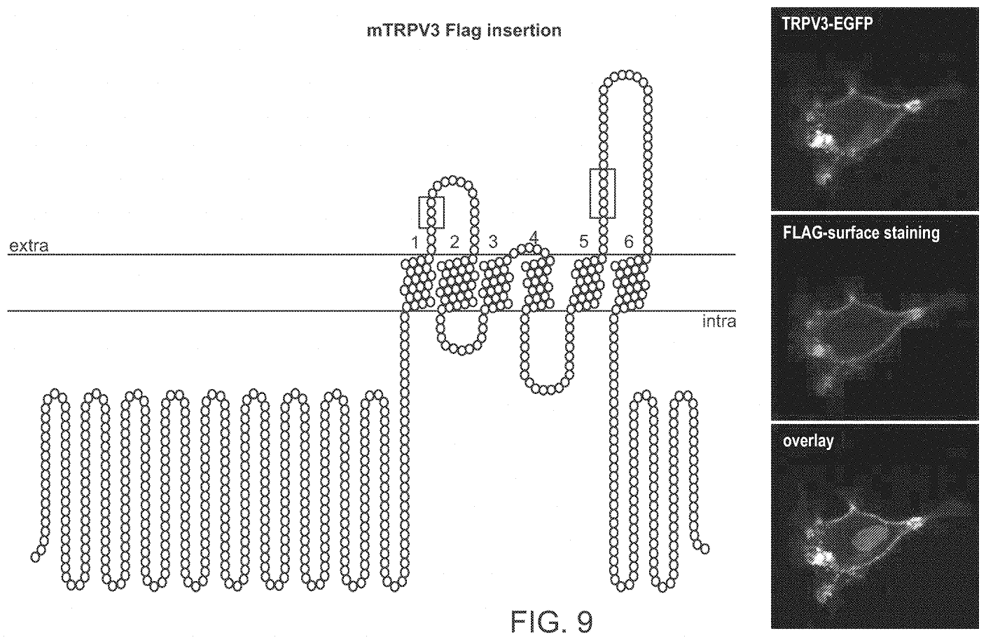

FIG. 9 shows a Procter visualization of mTRPV3 FLAG insertion. The right panels show images of surface FLAG, GFP-TRPV3, and an overlay.

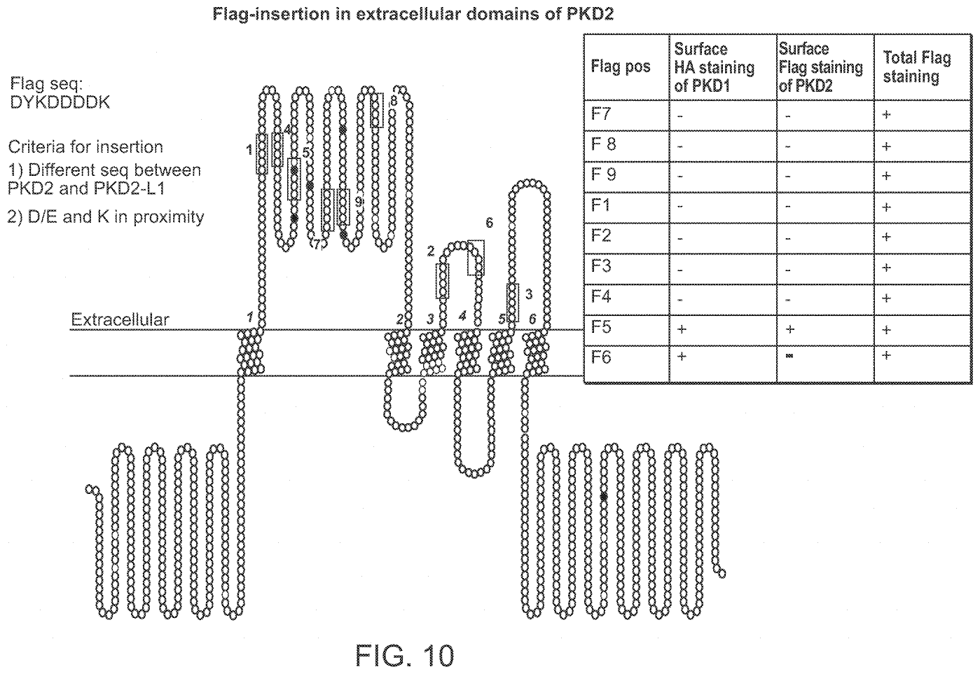

Flag insertion in extracellular domain of PKD2 using Procter visualization software is shown in FIG. 10. Nine positions were tested with only one resulting in surface FLAG staining. The criteria for insertion were 1) different sequences between PKD2 and PKD2-L1 and 2) D/E and K in proximity. DYKDDDDK is SEQ ID NO: 42

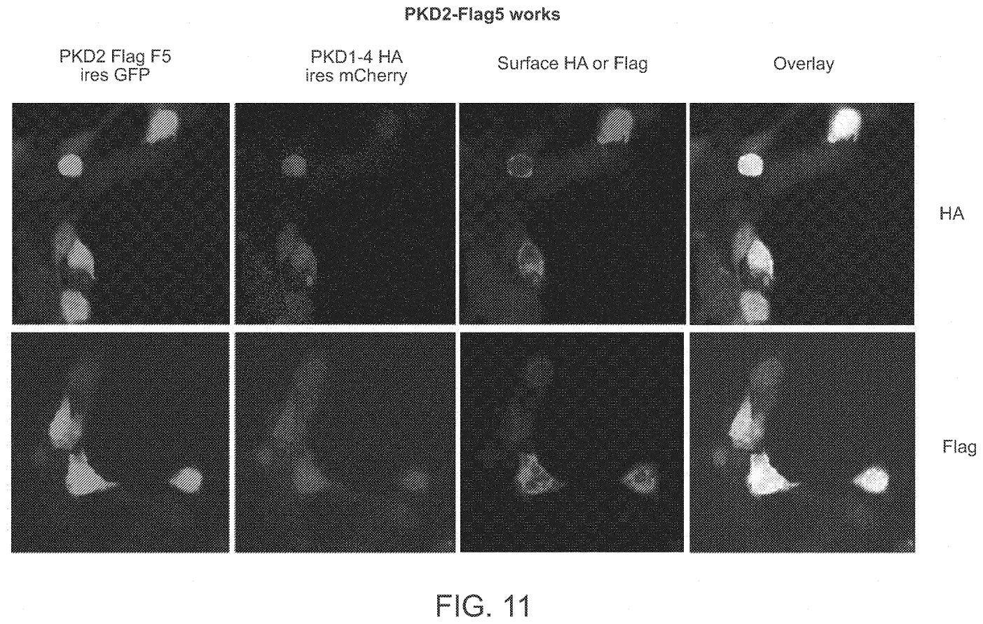

PKD2-FLAG5 was shown to work. FIG. 11 shows that upon co-transfection of P2Y12-PKD1-4 with PKD2-FLAG5 both epitopes are recognized in the plasma membrane using anti-HA and anti-FLAG antibodies.

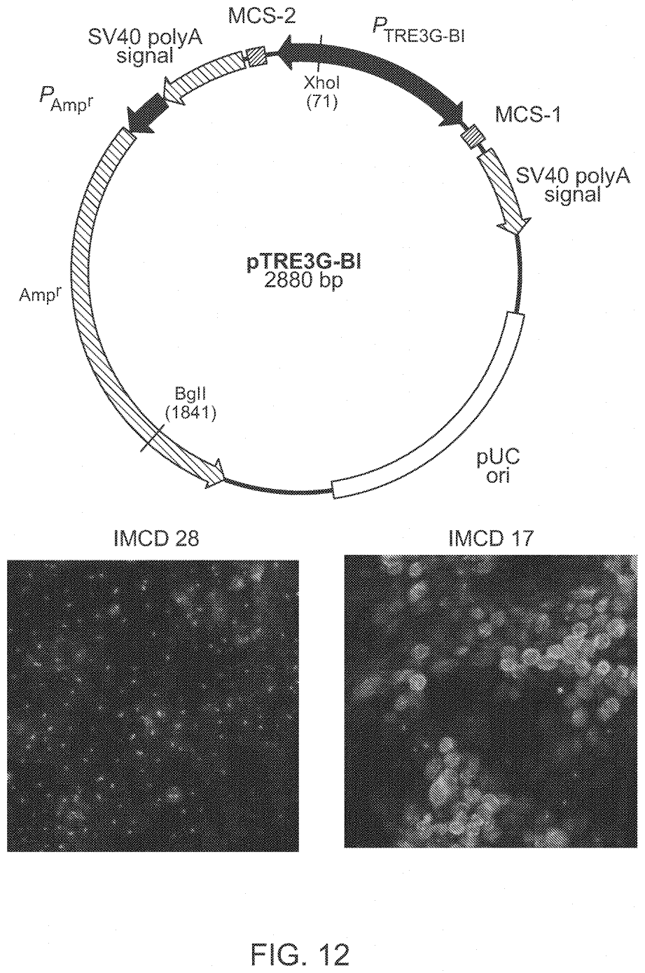

FIG. 12 shows pTRE3G-BI and images of IMCD 28 and IMCD 17. Stable cell lines were generated. Reliable co-expression of two proteins was required, A Tet-inducible dual promoter was used (Tet-on HEK cell line, Clontech). A Tet activator expressing mIMCD3 cell line was generated as well. Tet-on cell clones with best and worst cilia were selected: IMCD 17 and IMCD 28.

FIG. 13 shows schematic drawing of assay to detect PKD1 and PKD2 in the plasma membrane and primary cilium. HA and FLAG tag are visualized in the plasma membrane by incubating live cells with fluorescent dye conjugated anti-HA and anti-FLAG antibodies. Thus only membrane inserted PKD1/PKD2 proteins are immunolabeled, while intracellular localized proteins are not detected. On the right: HEK 3G cells (top) and mIMCD3 (bottom) stable clones express P2Y12-PKD1-4+PKD2-FLAG5 after 24 hour doxycycline induction are shown. While PKD1 and PKD2 localize to the plasma membrane in HEK cells both proteins traffic to the primary cilium in mIMCD3 cells. FIG. 14 shows examples of pathogenic point mutations in PKD1 causing ADPKD using the assay shown in FIG. 13. 5 randomly chosen mutations in PKD1 affect surface trafficking of the PKD1/PKD2 complex to various degrees.

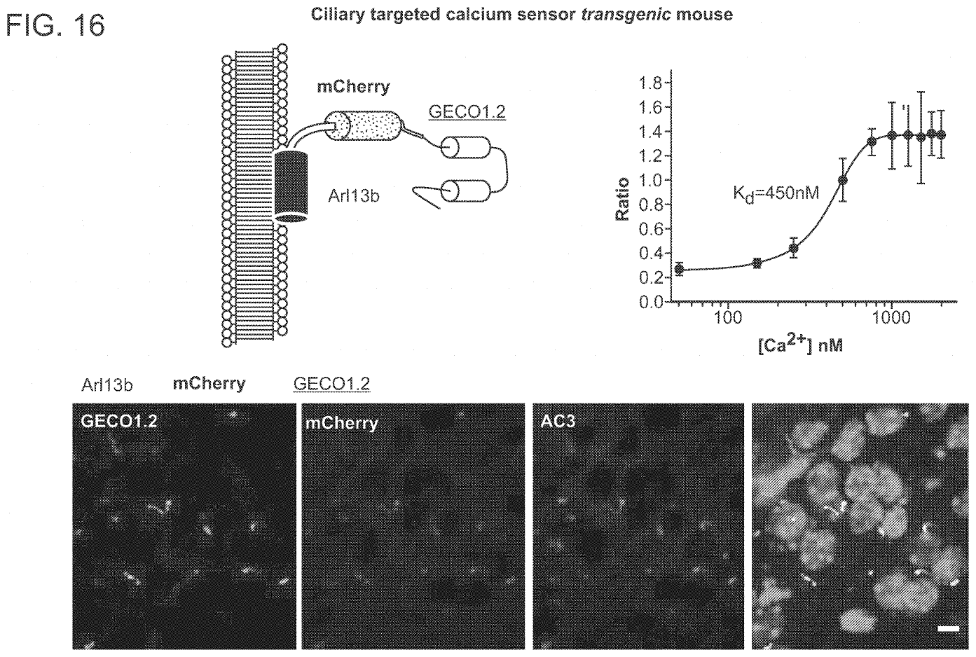

FIG. 15 shows examples of pathogenic point mutations in PKD2 causing ADPKD using the assay shown in FIG. 13. 5 randomly chosen mutations in PKD2 all abolish surface trafficking of the PKD1/PKD2 complex. A ciliary targeted calcium sensor transgenic mouse model is seen in FIG. 16.

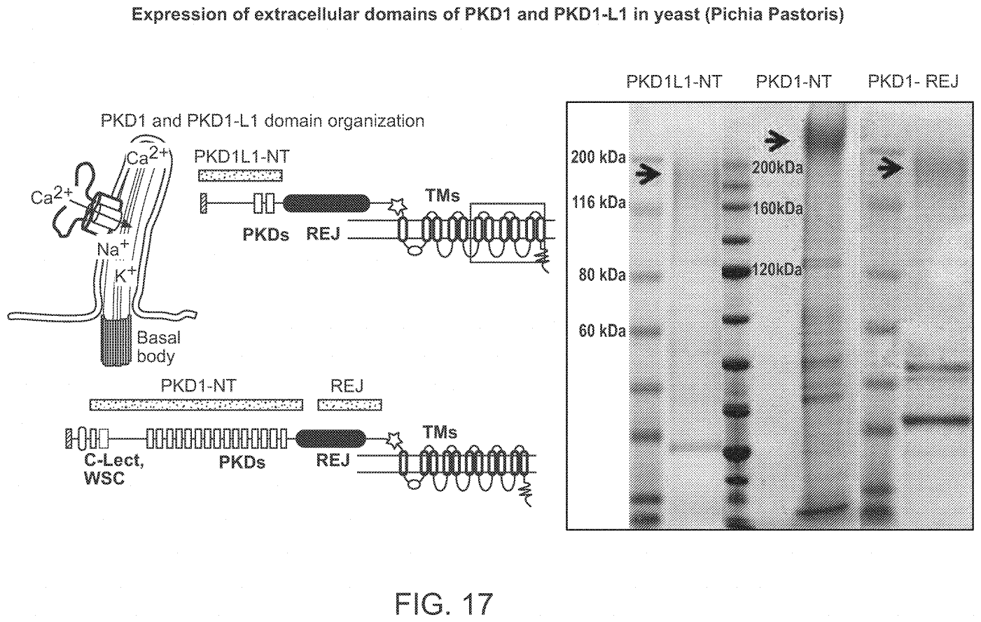

FIG. 17 shows expression of extracellular domains of PKD1 and PKD1-L1 in yeast (Pichia pastoris). The Pichia Pastoris expression system, which is commercially available from Life Technologies, was used for expression of the protein fragments. Nickel columns were used to purify the expressed proteins. The three purified proteins, are shown in FIG. 17, right panel. The DNA sequence of PKD1 extracellular fragments are shown in SEQ ID NOs: 14, 16 and 18. The protein sequences are shown in SEQ ID NOs: 15, 17, and 19.

The DNA sequence of PKD1 extracellular fragment (domains PKDI-PKDX) is shown in SEQ ID NO: 14. The DNA sequence was cloned into expression vector pPIC9 (life Technologies) for expression and purification in Pichia Pastoris. His tag used for purification is underlined.

The protein sequence of PKD1 extracellular fragment (domains PKDI-PKDX) expressed in Pichia Pastoris using pPIC9 expression vector (Life Technologies) is shown in SEQ ID NO: 15. His tag used for purification is underlined,

The DNA sequence of PKD1 extracellular fragment (PKD domain XII and REJ). DNA sequence (shown in SEQ ID NC): 16) was cloned in to expression vector pPIC9 (life Technologies) for expression and purification in Pichia Pastoris. His tag used for purification is underlined.

The protein sequence of PKD1 extracellular fragment (PKD domain XII and REJ) expressed in Pichia Pastoris using pPIC9 expression vector (Life Technologies) is shown in SEQ ID NO: 17. His tag used for purification is underlined.

The DNA sequence of PKD1L1 extracellular fragment. DNA sequence was cloned into expression vector pPIC9 (Life Technologies) for expression and purification in Pichia Pastoris is shown in SEQ ID NO: 18. His tag used for purification is underlined.

The protein sequence of PKD1 extracellular fragment (domains PKDI-PKDX) expressed in Pichia Pastoris using pPIC9 expression vector (Life Technologies) is shown in SEQ ID NO: 19. His tag used for purification is underlined.

Each of the sequences described herein are intended to designate the sequence with or without the presence of a FLAG or His tag and may be claimed with or without the FLAG or His tag (underlined sequence).

### Sequences 1-19:

hPKD2 DNA sequence with extracellular FLAG tag
(SEQ ID NO: 1)
atggtgaactccagtcgcgtgcagcctcagcagccggggacgccaagcggccgcccgcgccccgcgcgccggacccgg
gccggcgatggctggctgcgcggccgtgggcgccagcctcgccgccccgggcggcctctgcgagcagcggggcctgga
gatcgagatgcagcgcatccggcaggcggccgcgcgggaccccccggccggagccgcggcctccccttctcctccgctctc
gtcgtgctcccggcaggcgtggagccgcgataaccccggcttcgaggccgaggaggaggaggaggaggtggaaggggaa
gaaggcggaatggtggtggagatggacgtagagtggcgcccgggcagccggaggtcggccgcctcctcggccgtgagctc
cgtgggcgcgcggagccgggggcttgggggctaccacggcgcgggccacccgagcgggaggcggcgccggcgagagg
accagggcccgccgtgccccagcccagtcggcggcggggacccgctgcatcgccacctccccctggaagggcagccgcc
ccgagtggcctgggcggagaggctggttcgcgggctgcgaggtctctggggaacaagactcatggaggaaagcagcactaa
ccgagagaaataccttaaaagttttacgggaactggtcacatacctccttttctcatagtcttgtgcatcttgacctacggcatga
tgagctccaatgtgtactactacacccggatgatgtcacacctcttcctagacacccccgtgtccaaaacggagaaaactaacta
aaaactcgtcttccatggaagacttctggaagttcacagaaggctccttattggatgggctgtactggaagatgcagcccagca
accagactgactacaaagacgatgacgacaagagtttcatcttctatgagaacctgcgttaggggttccacgaatacggcaact
ccgagtcagaaatggatcctgctctatcccccaggacttgagagatgaaattaaagagtgctatgatgtctactctgtcagtagtg
aagatagggctccctttgggccccgaaatggaaccgcttggatctacacaagtgaaaaagacttgaatggtagtagccactggg
gaatcattgcaacttatagtggagctggctattatctggatttgtcaagaacaagagaggaaacagctgcacaagttgctagcctc
aagaaaaatgtctggctggaccgaggaaccagggcaacttttattgacttctcagtgtacaacgccaacattaacctgttctgtgt
ggtcaggttattggttgaattcccagcaacaggtggtgtgattccatcttggcaatttcagcctttaaagctgatccgatatgtcaca
acttttgatttcttcctggcagcctgtgagattatcttttgtttctttatcttttactatgtggtggaagagatattggaaattcgcattcac
aaactacactatttcaggagtttctggaattgtctggatgttgtgatcgttgtgctgtcagtggtagctataggaattaacatatacag
aacatcaaatgtggaggtgctactacagtttctggaagatcaaaatactttccccaactttgagcatctggcatattggcagataca
gttcaacaatatagctgctgtcacagtattttttgtctggattaagctcttcaaattcatcaattttaacaggaccatgagccagctctc
gacaaccatgtctcgatgtgccaaagacctgtttggctttgctattatgttcttcattattttcctagcgtatgctcagttggcatactt
gtctttggcactcaggtcgacttcagtactttccaagagtgtatcttcactcaattccgtatcattttgggcgatatcaactttgca
gagattgaggaagctaatcgagttttgggaccaatttatttcactacatttgtgttctttatgttcttcattcttttgaatatgtttttggcta
tcatcaatgatacttactctgaagtgaaatctgacttggcacagcagaaagctgaaatggaactctcagatcttatcagaaagggc
taccataaagctttggtcaaactaaaactgaaaaaaaataccgtggatgacatttcagagagtctgcggcaaggaggaggcaag
ttaaactttgacgaacttcgacaagatctcaaagggaagggccatactgatgcagagattgaggcaatattcacaaagtacgacc
aagatggagaccaagaactgaccgaacatgaacatcagcagatgagagacgacttggagaaagagagggaggacctggatt
tggatcacagttctttaccacgtcccatgagcagccgaagtttccctcgaagcctggatgactctgaggaggatgacgatgaag
atagcggacatagctccagaaggaggggaagcatttctagtggcgtttcttacgaagagtttcaagtcctggtgagacgagtgg
accggatggagcattccatcggcagcatagtgtccaagattgacgccgtgatcgtgaagctagagattatggagcgagccaaa
ctgaagaggagggaggtgctgggaaggctgttggatggggtggccgaggatgaaaggagggtcgtgacagtgaaatccat
agggaacagatggaacggctagtacgtgaagagttggaacgctgggaatccgatgatgcagcttcccagatcagtcatggttta
ggcacgccagtgggactaaatggtcaacctcgccccagaagctcccgcccatcttcctcccaatctacagaaggcatggaagg
tgcaggtggaaatgggagttctaatgtccacgtatga
hPKD2 protein sequence with FLAG sequence (FLAG tag underlined)
(SEQ ID NO: 2)
mvnssrvqpqqpgdakrppaprapdpgrlmagcaavgaslaapgglceqrgleiemqrirqaaardppagaaaspsppl
sscsrqawsrdnpgfeaeeeeeevegeeggmvvemdvewrpgsrrsaassavssvgarsrglggyhgaghpsgrrrrre
dqgppcpspvgggdplhrhlplegqpprvawaerlvrglrglwgtrlmeesstnrekylksvlrelvtyllflivlciltygm
mssnvyyytrmmsqlfldtpvsktektnfktlssmedfwkftegslldglywkmqpsnqtdykddddksfifyenlllgv
prirqlrvrngscsipqdlrdeikecydvysvssedrapfgprngtawiytsekdlngsshwgiiatysgagyyldlsrtreet
aaqvaslkknvwldrgtratfidfsvynaninlfcvvrllvefpatggvipswqfqplkliryvttfdfflaaceiifcffifyyvv
eeileirihlklhyfrsfwncldvvivvlsvvaiginiyrtsnvevllqfledqntfpnfehlaywqiqfnniaavtvffvwiklf
kfinfnrtmsqlsttmsrcakdlfgfaimffiiflayaqlaylvfgtqvddfstfqeciftqfriilgdinfaeieeanrvlgpiyftt
fvffmffillnmflaiindtysevksdlaqqkaemelsdlirkgyhkalvklklkkntvddiseslrqgggklnfdelrqdlkg
kghtdaeicaiftkydqdgdqeltehehqqmrddlekeredldldhsslprpmssrsfprslddseedddedsghssrrrgsi
ssgvsyeefqvlvrrvdrmehsigsivskidavivkleimeraklkrrevlgrlldgvaederlgrdseihreqmerlvreele
rwesddaasqishglgtpvglngqprprssrpsssqstegmegaggngssnvhv*
DNA sequence of P2Y12/PKD1-3 that traffics the PKD1/PKD2 complex to the plasma
membrane (HA-tag in capital letters, P2Y12 N-terminus, underlined)
(SEQ ID NO: 3)
atgTACCCATACGATGTTCCAGATTACGCTcaagccgtcgacaacctcacctctgcgcctggtaacac
cagtctgtgcaccagactacaaaatcacccagaccgccttcggcgccagcctcttcgtgcccccaagccatgtccgctttgt
gtttcctgagccgacagcggatgtaaactacatcgtcatgctgacatgtgctgtgtgcctggtgacctacatggtcatggccgcc
atcctgcacaagctggaccagttggatgccagccggggccgcgccatccctttctgtgggcagcggggccgcttcaagtacg
agatcctcgtcaagacaggctggggccggggctcaggtaccacggcccacgtgggcatcatgctgtatggggtggacagcc
ggagcggccaccggcacctggacggcgacagagccttccaccgcaacagcctggacatcttccggatcgccaccccgcac
agcctgggtagcgtgtggaagatccgagtgtggcacgacaacaaagggctcagccctgccggttcctgcagcacgtcatcgt
cagggacctgcagacggcacgcagcgccttcttcctggtcaatgactggctttcggtggagacggaggccaacgggggcctg
gtggagaaggaggtgctggccgcgagcgacgcagcccttttgcgcttccggcgcctgctggtggctgagctgcagcgtggct
tctttgacaagcacatctggctctccatgggaccggccgcctcgtagccgtttcactcgcatccagagggccacctgctgcgt
tctcctcatctgcctcttcctgggcgccaacgccgtgtggtacggggcgttggcgactctgcctacagcacggggcatgtgtcc
aggctgagcccgctgagcgtcgacacagtcgctgttggcctggtgtccagcgtggttgtctatcccgtctacctggccatcattt
tctcttccggatgtcccggagcaaggctgggagcccgagcccacacctgccgggcagcaggtgctggacatcgacag
ctgcctggactcgtccgtgctggacagctccttcctcacgttctcaggcctccacgctgagcaggcctttgttggacagatgaag
agtgacttgtttctggatgattctaagagtctggtgtgctggccctccggcgagggaacgctcagttggccggacctgctcagtg
acccgtccattgtgggtagcaatctgcggcagctggcacggggccaggcgggccatgggctgggcccagaggaggacggc
ttctccctggccagcccctactcgcctgccaaatccttctcagcatcagatgaagacctgatccagaggtccttgccgagggg
gtcagcagcccagcccctacccaagacacccacatggaaacggacctgctcagcagccgtccagcactccEggggagaag
acagagacgctggcgctgcagaggctgggggagctggggccacccagcccaggcctgaactgggaacagccccaggcag
cgaggctgtccaggacaggactggtggagggtctgcggaagcgcctgctgccggcctggtgtgcctccctggcccacgggc
tcagcctgctcctggtggctgtggctgtggctgtctcagggtgggtgggtgcgagcttccccccgggcgtgagtgttgcgtggc
tcctgtccagcagcgccagcttcctggcctcattcctcggctgggagccactgaaggtcttgctggaagccctgtacttctcactg
gtggccaagcggctgcacccggatgaagatgacaccctggtagagagcccggctgtgacgcctgtgagcgcacgtggtccc
cgcgtacggccaccccacggctttgcactcttcctggccaaggaagaagcccgcaaggtcaagaggctacatggcatgctgc
ggagcctcctggtgtacatgctttttctgctggtgaccctgctggccagctatggggatgcctcatgccatgggcacgcctaccgt
ctgcaaagcgccatcaagcaggagctgcacagccgggccttcctggccatcacgcggtcgaggagctctggccatggatgg
cccacgtgctgctcgccctacgtccacgggaaccagtccagcccagagctggggcccccacggctgcggcaggtgcggcgctgc
aggaagcactctacccagaccctcccggccccagggtccacacgtgctcggccgcaggaggcttcagcaccagcgattacg
acgttggctgggagagtcctcacaatggctcggggacgtgggcctattcagcgccggatctgctgggggcatggtcctgggg
ctcctgtgccgtgtatgacagcgggggctacgtgcaggagctgggcctgagcctggaggagagccgcgaccggctgcgctt
cctgcagctgcacaactggctggacaacaggagccgcgctgtgttcctggagctcacgcgctacagcccggccgtggggctg
cacgccgccgtcacgctgcgcctcgagttcccggcggccggccgcgccctggccgccctcagcgtccgcccctttgcgctgc
gccgcctcagcgcgggcctctcgctgcctctgctcacctcggtgtgcctgctgctgttcgccgtgcacttcgccgtggccgagg
cccgtacttggcacagggaagggcgctggcgcgtgctgcggctcggagcctgggcgcggtggctgctggtggcgctgacg
gcggccacggcactggtacgcctcgcccagctgggtgccgctgaccgccagtggacccgtttcgtgcgcggccgcccgcgc
cgcttcactagcttcgaccaggtggcgcagctgagctccgcagcccgtggcctggcggcctcgctgctcttcctgcttttggtca
aggctgcccagcagctacgcttcgtgcgccagtggtccgtctttggcaagacattatgccgagctctgccagagctcctggggg
tcaccttgggcctggtggtgctcggggtagcctacgcccagctggccatcctgctcgtgtcttcctgtgtggactccctctggag
cgtggcccaggccctgttggtgctgtgccctgggactgggctctctaccctgtgtcctgccgagtcctggcacctgtcacccctg
ctgtgtgtggggctctgggcactgcggctgtggggcgccctacggctgggggctgttattctccgctggcgctaccacgccttg
cgtggagagctgtaccggccggcctgggagccccaggactacgagatggtggagttgttcctgcgcaggctgcgcctctgga
tgggcctcagcaaggtcaaggagttccgccacaaagtccgctttgaagggatggagccgctgccctctcgctcctccaggggc
tccaaggtatccccggatgtgcccccacccagcgctggctccgatgcctcgcacccctccacctcctccagccagctggatgg
gctgagcgtgagcctgggccggctggggacaaggtgtgagcctgagccctcccgcctccaagccgtgttcgaggccctgctc
acccagtttgaccgactcaaccaggccacagaggacgtctaccagctggagcagcagctgcacagcctgcaaggccgcagg
agcagccgggcgcccgccggatcttcccgtggcccatccccgggcctgcggccagcactgcccagccgccttgcccgggc
cagtcggggtgtggacctggccactggccccagcaggacaccccttcgggccaagaacaaggtccaccccagcagcactta
g
Protein sequence of P2Y12/PKD1-3 that traffics the PKD1/PKD2 complex to the plasma
membrane (HA-tag + P2Y12 N-terminus, underlined)
(SEQ ID NO: 4)
MYPYDVPDYAQAVDNLTSAPGNTSLCTRDYKITQTAFGASLFVPPSHVRFVFPE
PTADVNYIVMLTCAVCLVTYMVMAAILHKLDQLDASRGRAIPFCGQRGRFKYEI
LVKTGWGRGSGTTAHVGIMLYGVDSRSGHRHLDGDRAFHRNSLDIFRIATPHSL
GSVWKIRVWHDNKGLSPAWFLQHVIVRDLQTARSAFFLVNDWLSVETEANGGL
VEKENLAASDAALLRFRRLLVAELQRGFFDKHIWLSIWDRPPRSRFTRIQRATCC
VLLICLFLGANAVWYGAVGDSAYSTGHVSRLSPLSVDTVAVGLVSSVVVYPVY
LAILFLFRMSRSKVAGSPSPTPAGQQVLDIDSCLDSSVLDSSFLTFSGLHAEQAFV
GQMKSDLFLDDSKSLVCWPSGEGTLSWPDLLSDPSIVGSNLRQLARGQAGHGL
GPEEDGFSLASPYSPAKSFSASDEDLIQQVLAEGVSSPAPTQDTHMETDLLSSLSS
TPGEKTETLALQRLGELGPPSPGLNWEQPQAARLSRTGLVEGLRKRLLPAWCAS
LAHGLSLLLVAVAVAVSGWVGASFPPGVSVAWLLSSSASFLASFLGWEPLKVLL
EALYFSLVAKRLHPDEDDTLVESPAVTPVSARVPRVRPPHGFALFLAKEEARKV
KRLHGMLRSLLVYMLFLLVTLLASYGDASCHGHAYRLQSAIKQELHSRAFLAIT
RSEELWPWMAHVLLPYVHGNQSSPELGPPRLRQVRLQEALYPDPPGPRVHTCSA
AGGFSTSDYDVGWESPHNGSGTWAYSAPDLLGAWSWGSCAVYDSGGYVQELG
LSLEESRDRLRFLQLHNWLDNRSRAVFLELTRYSPAVGLHAAVTLRLEFPAAGR
ALAALSVRPFALRRLSAGLSLPLLTSVCLLLFAVHFAVAEARTWHREGRWRVLR
LGAWARWLLLVALTAATALVRLAQLGAADRQWTRFVRGRPRRFTSFDQVAQLS
SAARGLAASLLFLLCVKAAQQLRFVRQWSVFGKTLCRALPELLGVTLGLVVLG
VAYAQLAILLVSSCVDSLWSVAQALLVLCPGTGLSTLCPAESWHLSPLLCVGLW
ALRLWGALRLGAVILRWRYHALRGELYRPAWEPQDYEMVELFLRRLRLWMGL
SKVKEFRHKVRFEGMEPLPSRSSRGSKVSPDVPPPSAGSDASHPSTSSSQLDGLSV
SLGRLGTRCEPEPSRLQAVFEALLTQFDRLNQATEDVYQLEQQLHSLQGRRSSR
APAGSSRGPSPGLRPALPSRLARASRGVDLATGPSRTPLRAKNKVHPSST.
DNA sequence of P2Y12/PKD1-4 that targets the PKD1/PKD2 complex to the plasma
membrane (HA-tag in capital letters. P2Y12 N-terminus underlined)
(SEQ ID NO: 5)
atgTACCCATACGATGTTCCAGATTACGCTcaagccgtcgacaacctcacctctgcgcctggtaacac
cagtctatgcaccagagactacaaaatcacccaggtccgctttgtgtttcctgagccgacagcggatgtaaactacatcgtcatg
ctgacatgtgctgtgtgcctggtgacctacatggtcatggccgccatcctgcacaagctggaccagttggatgccagccggggc
cgcgccatccctttctgtgggcagcggggccgcttcaagtacgagatcctcgtcaagacaggctggggccggggctcaggta
ccacggcccacgtgggcatcatgctgtatggggtggacagccggagcggccaccggcacctggacggcgacagagccttc
caccgcaacagcctggacatcttccggatcgccaccccgcacagcctgggtagcgtggaagatccgagtgtggcacgaca
acaaagggctcagccctgcctggttcctgcagcacgtcatcgtcagggacctgcagacggcacgcagcgccttcttcctggtc
aatgactggctttcggtggagacggaggccaacgggggcctggtggagaaggaggtgctggccgcgagcgacgcagccct
tttgcgcttccggcgcctgctggtggctgagctgcagcgtggcttctttgacaagcacatctggctctccatatgggaccggccg
cctcgtagccgtttcactcgcatccagagggccacctgctgcgttctcctcatctgcctcttcctgggcgccaacgccgtggta
cggggctgttggcgactctgcctacagcacggggcatgtgtccaggctgagcccgctgagcgtcgacacagtcgctgttggc
ctggtgtccagcgtggttgtctatcccgctacctggccatcctttttctcttccggatgtcccggagcaaggtggctgggagccc
gagccccacacctgccgggcagcaggtgctggacatcgacagctgcctggactcgtccgtgctggacagctccttcctcacgt
tctcaggcctccacgctgagcaggcctttgttggacagatgaagagtgacttgtttctggatgattctaagagtcggtgtgctggc
cctccggcgagggaacgctcagttggccggacctgctcagtgacccgtccattgtgggtagcaatctgcggcagctggcacg
gggccaggcgggccatgggctgggcccagaggaggacggcttctccctggccagcccctactcgcctgccaaatccttctca
gcatcagatgaagacctgatccagcaggtccttgccgagggggtcagcagcccagcccctacccaagacacccacatggaa
acggacctgctcagcagcctgtccagcactcctggggagaagacagagacgctggcgctgcagaggctgggggagctggg
gccacccagcccaggcctgaactgggaacagccccaggcagcgaggctgtccaggacaggactggtggagggtctgcgg
aacgcctgctgccggcctggtgtgcctccctggcccacgggctcagcctgctcctggtggctgtggctgtggctgtctcagg
gtgggtgggtgcgagcttccccccgggcgtgagtgttgcgtggctcctgtccagcagcgccagcttcctggcctcattcctcgg
ctgggagccactgaaggtcttgctggaagccctgtacttctcactggtggccaagcggctgcacccggatgaagatgacaccc
tggtagagagcccggctgtgacgcctgtgagcgcacgtgtgccccgcgtacggccaccccacggctttgcactcttcctggcc
aaggaagaagcccgcaaggtcaagaggctacatggcatgctgcggagcctcctggtgtacatgctttttctgctggtgaccctg
ctggccagctatggggatgcctcatgccatgggcacgcctaccgtctgcaaagcgccatcaagcaggagctgcacagccgg
gccttcctggccatcacgcggtctgaggagctctggccatggatggcccacgtgctgctgccctacgtccacgggaaccagtc
cagcccagagctggggcccccacggctgcggcaggtgcggctgcaggaagcactctacccagaccctcccggccccagg
gtccacacgtgctcggccgcaggaggcttcagcaccagcgattacgacgttggctgggagagtcctcacaatggctcgggga
cgtgggcctattcagcgccggatctgctgggggcatggtcctggggctcctgtgccgtgtatgacagcgggggctacgtgcag
gagctgggcctgagcctggaggagagccgcgaccggctgcgcttcctgcagctgcacaactggctggacaacaggagccg
cgctgtgttcctggagctcacgcgctacagcccggccgtggggctgcacgccgccgtcacgctgcgcctcgagttcccggcs
gccggccgcgccctggccgccctcagcgtccgcccctttgcgctgcgccgcctcagcgcgggcctctcgctgcctctgctca
cctcggtgtgcctgctgctgttcgccgtgcacttcgccgtggccgaggcccgtacttggcacagggaagggcgctggcgcgtg
ctgcggctcggagcctgggcgcggtggctgctggtggcgctgacggcggccacggcactggtacgcctcgcccagctgggt
gccgctgaccgccagtggacccgtttcgtgcgcggccgcccgcgccgcttcactagcttcgaccaggtggcgcagctgagct
ccgcagcccgtggcctggcggcctcgctgctcttcctgcttttggtcaaggctgcccagcagctacgcttcgtgcgccagtggt
ccgtctttggcaagacattatgccgagctctgccagagctcctgggggtcaccttgggcctggtggtgctcggggtagcctacg
cccagctggccatcctgctcgtgtcttcctgtgtggactccctctggagcgtggcccaggccctgttggtgctgtgccctgggac
tgggctctctaccctgtgtcctgccgagtcctggcacctgtcacccctgctgtgtgtggggctctgggcactgcggctgtggggc
gccctacggctgggggctgttattctccgctggcgctaccacgccttgcgtggagagctgtaccggccggcctgggagcccca
ggactacgagatggtggagttgttcctgcgcaggctgcgcctctggatgggcctcagcaaggtcaaggagttccgccacaaag
tccgctttgaagggatggagccgctgccctctcgctcctccaggggctccaaggtatccccggatgtgcccccacccagcgct
ggctccgatgcctcgcacccctccacctcctccagccagctggatgggctgagcgtgagcctgggccggctggggacaaggt
gtgagcctgagccctcccgcctccaagccgtgttcgaggccctgctcacccagtttgaccgactcaaccaggccacagagga
cgtctaccagctggagcagcagctgcacagcctgcaaggccgcaggagcagccgggcgcccgccggatcttcccgtggcc
catccccgggcctgcggccagcactgcccagccgccttgcccgggccagtcggggtgtggacctggccactggccccagca
ggacaccccttcgggccaagaacaaggtccaccccagcagcacttag
Protein sequence of P2Y12/PKD1-4 that targets the PKD1/PKD2 complex to the plasma
membrane (HA-tag + P2Y12 N-terminus, underlined)
(SEQ ID NO: 6)
MYPYDVPDYAQAVDNLTSAPGNTSLCTRDYKITQVRFVFPEPTADVNYIVMLTC
AVCLVTYMVMAAILHKLDQLDASRGRAIPFCGQRGRFKYEILVKTGWGRGSGT
TAHVGIMLYGVDSRSGHRHLDGDRAFHRNSLDIFRIATPHSLGSVWKIRVWHDN
KGLSPAWFLQHVIVRDLQTARSAFFLVNDWLSVETEANGGLVEKEVLAASDAA
LLRFRRLLVAELQRGFFDKHIWLSIWDRPPRSRFTRIQRATCCVLLICLFLGANAV
WYGAVGDSAYSTGHVSRLSPLSVDTVAVGLVSSVVVYPVYLAILFLFRMSRSKV
AGSPSPTPAGQQVLDIDSCLDSSVLDSSFLTFSGLHAEQAFVGQMKSDLFLDDSK
SLVCWPSGEGTLSWPDLLSDPSIVGSNLRQLARGQAGHGLGPEEDGFSLASPYSP
AKSFSASDEDLIQQVLAEGVSSPAPTQDTHMETDLLSSLSSTPGEKTETLALQRL
GELGPPSPGLNWEQPQAARLSRTGLVEGLRKRLLPAWCASLAHGLSLLLVAVA
VAVSGWVGASFPPGVSVAWLLSSSASFLASHFLGWEPLKVLLEALYFSLVAKRLH
PDEDDTLVESPAVTPVSARVPRVRPPHGFALFLAKEEARKVKRLHGMLRSLLVY
MLFLLVTLLASYGDASCHGHAYRLQSAIKQELHSRAFLAITRSEELWPWMAHVL
LPYVHGNQSSPELGPPRLRQYRLQEALYPDPPGPRVHTCSAAGGFSTSDYDVGW
ESPHNGSGTWAYSAPDLLGAWSWGSCAVYDSGGYVQELGLSLEESRDRLRFLQ
LHNWLDNRSRAVFLELTRYSPAVGLHAAVTLRLEFPAAGRALAALSVRPFALRR
LSAGLSLPLLTSVCLLLFAVHFAVAEARTWHREGRWRVLRLGAWARWLLVALT
AATALVRLAQLGAADRQWTRFVRGRPRRFTSFDQVAQLSSAARGLAASLLFLL
LVKAAQQLRFVRQWSVFGKTLCRALPELLGVTLGLVVLGVAYAQLAILLVSSC
VDSLWSVAQALLVLCPGTGLSTLCPAESWHLSPLLCVGLWALRLWGALRLGAV
ILRWRYHALRGELYRPAWEPQDYEMVELFLRRLRLWMGLSKVKEFRHKVRFE
GMEPLPSRSSRGSKVSPDVPPPSAGSDASHPSTSSSQLDGLSVSLGRLGTRCEPEP
SRLQAVFEALLTQFDRLNQATEDVYQLEQQLHSLQGRRSSRAPAGSSRGPSPGL
RPALPSRLARASRGVDLATGPSRTPLRAKNKVHPSST.
DNA sequence of PKD1 truncation that traffics the PKD1/PKD2 complex to the plasma
membrane
(SEQ ID NO: 7)
atgaagcggctgcacccggatgaagatgacaccctggtagagagcccggctgtgacgcctgtgagcgcacgtgtgccccgc
gtacggccaccccacggctttgcactcttcctggccaaggaagaagcccgcaaggtcaagaggctacatggcatgctgcgga
gcctcctggtgtacatgctttttctgctggtgaccctgctggccagctatggggatgcctcatgccatgggcacgcctaccgtctg
caaagcgccatcaagcaggagctgcacagccgggccttcctggccatcacgcggtctgaggagctctggccatggatggcc
cacgtgctgctgccctacgtccacgggaaccagtccagcccagagcggggcccccacggctgcggcaggtgcggctgca
ggaagcactctacccagaccctcccggccccagggtccacacgtgctcggcgcaggaggcttcagcaccagcgattacga
cgttggctgggagagtcctcacaatggctcggggacgtgggcctattcagcgccggatctgctgggggcatggtcctggggct
cctgtgccgtgtatgacagcgggggctacgtgcaggagctgggcctgagcctggaggagagccgcgaccggctgcgcttcc
tgcagctgcacaactggctggacaacaggagccgcgctgttcctggagctcacgcgctacagcccggccgtggggctgca
cgccgccgtcacgctgcgcctcgagttcccggcggccggccgcgccctggccgccctcagcgtccgcccctttgcgctgcg
ccgcctagcgcgggcctctcgctgcctctcacctcggtgtgcctgctgttcgccgtgcacttcgccgtggccgaggc
ccgtacttggcacagggaagggcgctggcgcgtgctgcggctcggagcctgggcgcggtggctgctggtggcgctgacgg
cggccacggcactggtacgcctcgcccagctgggtgccgctgaccgccagtggacccgtttcgtgcgcggccgcccgcgcc
gcttcactagcttcgaccaggtggcgcagctgagctccgcagcccgtggcctggcggcctcgctgctcttcctgcttttggtcaa
ggctgcccagcagctacgcttcgtgcgccagtggtccgtctttggcaagacattatgccgagctctgccagagctcctgggggt
caccttgggcctggtggtgctcggggtagcctacgcccagctggccatcctgctcgtgtcttcctgtgtggactccctaggagc
gtggcccaggccctgttggtgctgtgccctgggactgggctctctaccctgtgtccgtgccgagtcctggcacctgtcacccctgc
tgtgtgtggggctctgggcactgcggctgtggggcgccctacggctgggggctgttattctccgctggcgctaccacgccttgc
gtggagagctgtaccggccggcctgggagccccaggactacgagatggtggagttgttcctgcgcaggctgcgcctctggat
gggcctcagcaaggtcaaggagttccgccacaaagtccgctttgaagggatggagccgctgccctctcgctcctccaggggct
ccaaggtatccccggagtgacccccacccagcgctggctccgatgcctcgcaccctaccacctcctccagccagctggatggg
ctgagcgtgagcctgggccggctggggacaaggtgtgagcctgagccctcccgcctccaagccgtgttcgaggccctgctca
cccagtttgaccgactcaaccaggccacagaggacgtctaccagctggagcagcagctgcacagcctgcaaggccgcagga
gcagccgggcgcccgccggatcttcccgtggcccatccccgggcctgcggccagcactgcccagccgccttgcccgggcc
agtcggggtgtggacctggccactggccccagcaggacaccccttcgggccaagaacaaggtccaccccagcagcacttag
Protein sequence of a PKD1 truncation that traffics the PKD1/PKD2 complex to the
plasma membrane
(SEQ ID NO: 8)
mkrlhpdeddtlvespavtpvsarvprvrpphgfalflakeearkvkrlhgmlrsllvymlfllvtllasygdaschghayrl
qsaikqelhsraflaitrseelwpwmahvllpyvhgnqsspelgpprlrqvrlqealypdppgprvhicsaaggfstsdydv
gwesphngsgtwaysapdllgawswgscavydsggyvqelglsleesrdrlrflqlhnwldnrsravfleltryspavglh
aavtlrlefpaagralaalsvrpfalrrlsaglslplltsvclllfavhfavaeartwhregrwrvlrlgawarwllvaltaatalvrl
aqlgaadrqwtrfvrgrprrftsfdqvaqlssaarglaasllflllvkaaqqlrfvrqwsvfgktlcralpellgvtlglvvlgvay
aqlaillvsscvdslwsvaqallvlcpgtglstlcpaeswhlspllcvglwalrlwgalrlgavilrwryhalrgelyrpawepq
dyemvelflrrlrlwmglskvkefrhkvrfegmeplpsrssrgskvspdvpppsagsdashpstsssqldglsyslgrlgtre
epepsrlqavfealltqfdrlnqatedvyqleqqlhslqgrrssrapagssrgpspglrpalpsrlarasrgvdlatgpsrtplrak
nkvhpsst*
HA-tag in capital letters, P2Y12 N-terminus
(SEQ ID NO: 9)
atgTACCCATACGATGTTCCAGATTACGCTcaagccgtcgacaacctcacctctgcgcctggtaacac
cagtctGtgcaccagagactacaaaatcacccag
HA-tag + P2Y12 N-terminus
(SEQ ID NO: 10)
MYPYDVPDYAQAVDNLTSAPGNTSLCTRDYKITQ
hPKD2 protein sequence without FLAG seq.
(SEQ ID NO: 11)
mvnssrvqpqqpgdakrppaprapdpgrlmagcaavgaslaapgglceqrgleiemqrirqaaardppagaaaspsppl
sscsrqawsrdnpgfeaeeeeeevegeeggmvvemdvewrpgsrrsaassavssvgarsrglggyhgaghpsgrrrrrre
dqgppcpspvgggdplhrhlplegqpprvawaerlvrglrglwgtrlmeesstnrekylksvlrelvtyllflivlciltygm
mssnvyyytrmmsqlfldtpvsktektnfktlssmedfwkftegslldglywkmqpsnqtsfifyenlllgvprirqlrvrn
gscsipqdlrdekecydvysvssedrapfgprngtawiytsekdlngsshwgiiatysgagyyldlsrtreetaaqvaslkk
nvwkldrgtratfidfsvynaninlfcvvrllvefpatggvipswqfqplkliryvttfdfflaaceiifcffifyyvveeileirihk
lhyfrsfwncldvvivvlsvvaiginiyrtsnvevllqfledqntfpnfehlaywqiqfnniaavtvffwiklfinfnrtms
qlsttmsrcakdlfgfaimffiiflayaqlaylvfgtqvddfstfqeciftqfriilgdinfaeieeanrvlgpiyfttfvffmffilln
mflaiindtysevksdlaqqkaemelsdlirkgyhkalvklklkkntvddiseslrqgggklnfdelrqdlkgkghtdaica
iftkydqdgdqeltehehqqmrddlekeredldldhsslprpmssrsfprslddseedddedsghssrrrgsissgvsyeef
qvlvrrvdrmehsigsivskidavivkleimeraklkrrevlgrlldgvaederlgrdseihreqmerlvreelerwesddaa
sqishglgtpvglngqprprssrpsssqstegmegaggngssnvhv*
pTRE3G-Bi vector DNA sequence (Takara-Bioscience, Inc)
(SEQ ID NO: 12)
agcgatctgacggacactaaacgagctctgcttatataggcctcccaccgtacacgccacctcgacatactcgagtttactccct
atcagtgatagagaacgtatgaagagtttactccctatcagtgatagagaacgtatgcagactttactccctatcagtgatagaga
acgtataaggagtttactccctatcagtgatagagaacgtatgaccagtttactccctatcagtgatagagaacgtatctacagttta
ctccctatcagtgatagagaacgtatatccagtttactccctatcagtgatagagaacgtataagctttaggcgtgtacggtgggc
gcctataaaagcagagctcgtttagtgaaccgtcagatcgcctggagcaattccacaacacttttgtcttataccaactttccgtac
cacttcctaccctcgtaaagtcgacaccggggcccagatctccgcggggatccatcgatccgcggccgccggcgatatctcca
gaggatcataatcagccataccacatttgtagaggttttacttgctttaaaaaacctcccacacctccccctgaacctgaaacataa
aatgaatgcaattgttgttgttaacttgtttattgcagcttataatggttacaaataaagcaatagcatcacaaatttcacaaataaagc
atttttttcactgccccgagcttcctcgctcactgactcgctgcgctcggtcgttcggctgcggcgagcggtatcagctcactcaaa
ggcggtaatacggttatccacagaatcaggggataacgcaggaaagaacatgtgagcaaaaggccagcaaaaggccagga
accgtaaaaaggccgcgttgctggcgtttttccataggctccgcccccctgacgagcatcacaaaaatcgacgctcaagtcaga
ggtggcgaaacccgacaggactataaagataccaggcgtttccccctggaagctccctcgtgcgctctcctgttccgaccagc
cgcttaccggatacctgtccgcctttctcccttcgggaagcgtggcgctttctcaatgctcacgctgtaggtatctcagttcggtgta
ggtcgttcgctccaagctgggctgtgtgcacgaaccccccgttcagcccgaccgctgcgccttatccggtaactatcgtcttgag
tccaacccggtaagacacgacttatcgccactggcagcagccactggtaacaggattagcagagcgaggtatgtaggcggtg
ctacagagttcttgaagtggtggcctaactacggctacactagaaggacagtatttggtatctgcgctctgctgaagccagttacc
ttcggaaaaagagttggtagctcttgatccggcaaacaaaccaccgctggtagcggtggtttttttgtttgcaagcagcagattac
gcgcagaaaaaaaggatctcaagaagatcctttgatctttttctacggggtctgacgctcagtggaacgaaaactcacgttaaggg
attttggtcatgagattatcaaaaaggatcttcacctagatccttttaaattaaaaatgaagttttaaatcaatctaaagtatatatgagt
aaacttggtctgacagttaccaatgcttaatcagtgaggcacctatctcagcgatcgtctatttcgttcatccatagttgcctgactc
cccgtcgtgtagataactacgatacgggagggcttaccatctggccccagtgctgcaatgataccgcgagacccacgctcacc
ggctccagatttatcagcaataaaccagccagccggaagggccgagcgcagaagtggtcctgcaactttatccgcctccatcc
agtctattaattgttgccgggaagctagagtaagtagttcgccagttaatagtttgcgcaacgttgttgccattgctacaggcatcgt
ggtgtcacgctcgtcgtttggtatggcttcattcagctccggttcccaacgatcaaggcgagttacatgatcccccatgttgtgcaa
aaaagcggttagctccttcggtcctccgatcgttgtcagaagtaagttggccgcagtgttatcactcatggttatggcagcactgc
ataattctcttactgtcatgccatccgtaagatgcttttctgtgactggtgagtactcaaccaagtcattctgagaatagtgatgcgg
cgaccgagttgctcttgcccggcgtcaatacgggataataccgcgccacatagcagaactttaaaagtgctcatcattggaaaac
gttcttcggggcgaaaactctcaaggatcttaccgctgttgagatccagttcgatgtaacccactcgtgcacccaactgatcttca
gcatcttttactttcaccagcgtttctgggtgagcaaaaacaggaaggcaaaatgccgcaaaaaagggaataagggcgacacg
gaaatgttgaatactcatactcttcctttttcaatattattgaagcatttatcagggttattgtctcatgagcggatacatatttgaatgtat
ttagaaaaataaacaaataggggttccgcgcacatttccccgaaaagtgccacctgacgtcggcagtgaaaaaatgctttattt
gtgaaatttgtgatgctattgctttatttgtaaccattataagctgcaataaacaagttaacaacaacaattgcattcattttatgtttcag
gttcagggggaggtgtgggaggttttttaaagcaagtaaaacctctacaaatgtggtatggctgattatgatcctctagacatatgc
tgcagacgcgtggtacccccggggaattctccag.
DNA sequence of pEGFP backbone vector with ires mCherry
(SEQ ID NO: 13)
tagttattaatagtaatcaattacggggtcattagttcatagcccatatatggagttccgcgttacataacttacggtaaatggcccg
cctggctgaccgcccaacgacccccgcccattgacgtcaataatgacgtatgttcccatagtaacgccaatagggactttccatt
gacgtcaatgggtggagtatttacggtaaactgcccacttggcagtacatcaagtgtatcatatgccaagtacgccccctattgac
gtcaatgacggtaaatggcccgcctggcattatgcccagtacatgaccttatgggactttcctacttggcagtacatctacgtatta
gtcatcgctattaccatggtgatgcggattttggcagtacatcaatgggcgtggatagcggtttgactcacggggatttccaagtctc
caccccattgacgtcaatgggagtttgttttggcaccaaaatcaacgggactttccaaaatgtcgtaacaactccgccccattgac
gcaaatgggcggtaggcgtgtacggtgggaggtctatataagcagagctggtttagtgaaccgtcagatccgctagcgctacc
ggactcagatctcgagctcaagcttcgaattctgcagtcgacggtaccgcggcgctacgtaaattccgccccccccccccctaa
cgttactggccgaagccgcttggaataaggccggtgtgcgtttgtctatatgttattttccaccatattgccgtcttttggcaatgtga
gggcccggaaacctggccctgtcttcttgacgagcattcctaggggtctttcccctcycgccaaaggaatgcaaggtctgttgaa
tgtcgtgaaggaagcagttcctctggaagcttcttgaagacaaacaacgtctgtagcgaccctttgcaggcagcggaacccccc
acctggcgacaggtgcctctgcggccaaaagccacgtgtatmagatacacctgcaaaggcggcacaaccccagtgccacgt
tgtgagttggatagttgtggaaagagtcaaatggctctcctcaagcgtattcaacaaggggctgaaggatgcccagaaggtacc
ccattgtatgggatctgatctggggcctcgrtrcacatgctttacatgtgtttagtcgaggttaaaaaaacgtctaggccccccgaa
ccacggggacgtggttttcctttgaaaaacacgatgataatatggccamaammawratggtgagcaagggcgaggaggat
aacatggccatcatcaaggagttcatgcgcttcaaggtgcacatggagggctccgtgaacggccacgagttcgagatcgagg
gcgagggcgagggccgcccctacgagggcacccagaccgccaagctgaaggtgaccaagggtggccccctgcccttcgc
ctgggacatcctgtcccctcagttcatgtacggctccaaggcctacgtgaagcaccccgccgacatccccgactacttgaagct
gtccttccccgagggcttcaagtgggagcgcgtgagtgaacttcgaggacggcggcgtggtgaccgtgacccaggactcctcc
ctgcaggacggcgagttcatctacaaggtgaagctgcgcggcaccaacttcccctccgacggccccgtaatgcagaagaaga
ccatgggctgggaggcctcctccgagcggatgtaccccgaggacggcgccctgaagggcgagatcaagcagaggctgaag
ctgaaggacggcggccactacgacgctgaggtcaagaccacctacaaggccaagaagcccgtgcagctgcccggcgccta
caacgtcaacatcaagttggacatcacctcccacaacgaggactacaccatcgtggaacagtacgaacgcgccgagggccgc
cactccaccggcggcatggacgagctgtacaagtaagcggccgcgactctagatcataatcagccataccacatttgtagaggt
tttacttgctttaaaaaacctcccacacctccccctgaacctgaaacataaaatgaatgcaattgttgttgttaacttgtttattgcagc
ttataatggttacaaataaagcaatagcatcacaaatttcacaaataaagcatttttttcactgcattctagttgtggtttgtccaaactc
atcaatgtatcttaaggcgtaaattglaagcgttaatattttgttaaaattcgcgttaaatttttgttaaatcagctcattttttaaccaata
ggccgaaatcggcaaaatcccttataaatcaaaagaatagaccgagatagggttgagtgttgttccagtttggaacaagagtcca
ctattaaafgaacgtggactccaacgtcaaagggcgaaaaaccgtctatagggcgatggcccactacgtgaaccatcacccta
atcaagttttttggggtcgaggtgccgtaaagcactaaatcggaaccctaaagggagcccccgatttagagcttgacggggaaa
gccggcgaacgtggcgagaaaggaagggaagaaagcgaaaggagcgggcgctagggcgctggcaagtgtagcggtcac
gctgcgcgtaaccaccacacccgccgcgcttaatgcgccgctacagggcgcgtcaggtggcacttttcggggaaatgtgcgc
ggaacccctatttgtttatttttctaaatacattcaaatatgtatccgctcatgagacaataaccctgataaatgcttcaataatattgaa
aaaggaagagtcctgaggcggaaagaaccagctgtggaatgtgtgtcagttagggtgtggaaagtccccaggctccccagca
ggcagaagtatgcaaagcatgcatctcaattagtcagcaaccaggtgtggaaagtccccaggctccccagcaggcagaagtat
gcaaagcatgcatctcaattagtcagcaaccatagtcccgcccctaactccgcccatcccgcccctaactccgcccagttccgc
ccattctccgccccatggctgactaattttttttatttatgcagaggccgaggccgcctcggcctctgagctattccagaagtagtg
aggaggcttttttggaggcctaggcttttgcaaagatcgatcaagagacaggatgaggatcgtttcgcatgattgaacaagatgg
attgcacgcaggttctccggccgcttgggtggagaggctattcggctatgactgggcacaacagacaatcggctgctctgatgc
cgccgtgttccggctgtcagcgcaggggcgcccggttctttttgtcaagaccgacctgtccggtgccctgaatgaactgcaaga
cgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctg
atgcaatgcggcggctgcatacgcttgatccggctacctgcccattcgaccaccaagcgaaacatcgcatcgagcgagcacgt
actcggatggaagccggtcttgtcgatcaggatgatctggacgaagagcatcaggggctcgcgccagccgaactgttcgcca
ggctcaaggcgagcatgcccgacggcgaggatctcgtcgtgacccatggcgatgcctgcttgccgaatatcatggtggaaaat
ggccgcttttctggattcatcgactgtggccggctgggtgtggcggaccgctatcaggacatagcgttggctacccgtgatattg
ctgaagagcttggcggcgaatgggctgaccgcttcctcgtgctttacggtatcgccgctcccgattcgcagcgcatcgccttcta
tcgccttcttgacgagttcttctgagcgggactctggggttcgaaatgaccgaccaagcgacgcccaacctgccatcacgagatt
tcgattccaccgccgccttctatgaaaggttgggcttcggaatcgttttccgggacgccggctggatgatcctccagcgcgggg
atctcatgctggagttcttcgcccaccctagggggaggctaactgaaacacggaaggagacaataccggaaggaacccgcgc
tatgacggcaataaaaagacagaataaaacgcacggtgttgggtcgtttgttcataaacgcggggttcggtcccagggctggca
ctctgtcgataccccaccgagaccccattggggccaatacgcccgcgtttcttccttttccccaccccaccccccaagttcgggt
gaaggcccagggctcgcagccaacgtcggggcggcaggccctgccatagcctcaggttactcatatatactttagattgattta
aaacttcatttttaatttaaaaggatctaggtgaagatcctttttgataatctcatgaccaaaatcccttaacgtgagttttcgttccact
gagcgtcagaccccgtagaaaagatcaaaggatcttcttgagatcctttttttctgcgcgtaatctgctgcttgcaaacaaaaaaac
caccgctaccagcggtggtttgtttgccggatcaagagctaccaactctttttccgaaggtaactggcttcagcagagcgcagat
accaaatactgtccttctagtgtagccgtagttaggccaccacttcaagaactctgtagcaccgcctacatacctcgctctgctaat
cctgttaccagtggctgctgccagtggcgataagtcgtgtcttaccgggttggactcaagacgatagttaccggataaggcgca
gcggtcgggctgaacggggggttcgtgcacacagcccagcttggagcgaacgacctacaccgaactgagatacctacagcg
tgagctatgagaaagcgccacgcttcccgaagggagaaaggcggacaggtatccggtaagcggcagggtcggaacaggag
agcgcacgagggagcttccagggggaaacgcctggtatctttatagtcctgtcgggtttcgccacctctgacttgagcgtcgattt
ttgtgatgctcgtcaggggggcggagcctatggaaaaacgccagcaacgcggcctttttacggttcctggccttttgctggccttt
tgctcacatgttctttcctgcgttatcccctgattctgtggataaccgtattaccgccatgcat
DNA sequence of PKD1 extracellular fragment (domains PKDI-PKDX). DNA
sequence was cloned into expression vector pPIC9 (Life Technologies) for expression
and purification in Pichia Pastoris, His tag uscd for purification is underlined.
(SEQ ID NO: 14)
gccacacgctgggacttcggagacggctccgccgaggtggatgccgctgggccggctgcctcgcatcgctatgtg
ctgcctgggcgctatcacgtgacggccgtgctggccctgggggccggctcagccctgctggggacagacgtgcaggtggaa
gcggcacctgccgccctggagctcgtgtgcccgtcctcggtgcagagtgacgagagccttgacctcagcatccagaaccgcg
gtggttcaggcctggaggccgcctacagcatcgtggccctgggcgaggagccggcccgagcggtgcacccgctctgcccct
cggacacggagatcttccctggcaacgggcactgctaccgcctggtggtggagaaggcggcctggctgcaggcgcaggag
cagtgtcaggcctgggccggggccgccctggcaatggtggacagtcccgccgtgcagcgcttcctggtctcccgggtcacca
ggagcctagacgtgtggatcggcttctcgactgtgcagggggtggaggtgggcccagcgccgcagggcgaggccttcagcc
tggagagctgccagaactggctgcccggggagccacacccagccacagccgagcactgcgtccggctcgggcccaccgg
gtggtgtaacaccgacctgtgctcagcgccgcacagctacgtctgcgagctgcagcccggaggcccagtgcaggatgccga
gaacctcctcgtgggagcgcccagtggggacctgcagggacccctgacgcctctggcacagcaggacggcctctcagcccc
gcacgagcccgtggaggtcatggtattcccgggcctgcgtctgagccgtgaagccttcctcaccacggccgaatttgggaccc
aggagctccggcggcccgcccagctgcggctgcaggtgtaccggctcctcagcacagcagggaccccggagaacggcag
cgagcctgagagcaggtccccggacaacaggacccagctggcccccgcgtgcatgccagggggacgctggtgccctgga
gccaacatctgcttgccgctggacgcctcctgccacccccaggcctgcgccaatggctgcacgtcagggccagggctacccg
gggccccctatgcgctatggagagagttcctcttctccgttcccgcggggccccccgcgcagtactcggtcaccctccacggc
caggatgtcctcatgctccctggtgacctcgttggcttgcagcacgacgctggccctggcgccctcctgcactgctcgccggct
cccggccaccctggtccccaggccccgtacctctccgccaacgcctcgtcatggctgccccacttgccagcccagctggagg
gcacttgggcctgccctgcctgtgccctgcggctgcttgcagccacggaacagctcaccgtgctgctgggcttgaggcccaac
cctgaactgcggctgcctgggcgctatgaggtccgggcagaggtgggcaatggcgtgtccaggcacaacctctcctgcagct
ttgacgtggtctccccagtggctgggctgcgggtcatctaccctgccccccgcgacggccgcctctacgtgcccaccaacggc
tcagccttggtgctccaggtggactctggtgccaacgccacggccacggctcgctggcctgggggcagtgtcagcgcccgctt
tgagaatgtctgccctgccctggtggccaccttcgtgcccggctgcccctgggagaccaacgataccctgttctcagtggtagc
actgccgtggctcagtgagggggagcacgtggtggacgtggtggtggaaaacagcgccagccgggccaacctcagcctgc
gggtgacggcggaggagcccatctgtggcctccgcgccacgcccagccccgaggcccgtgtactgcagggagtcctagtga
ggtacagccccgtggtggaggccggctcggacatggtcttccggtggaccatcaacgacaagcagtccctgaccttccagaa
cgtggtcttcaatgtcatttatcagagcgcggcggtcttcaagctctcactgacggcctccaaccacgtgagcaacgtcaccgtg
aactacaacgtaaccgtggagcggatgaacaggatgcagggtctgcaggtctccacagtgccggccgtgctgtcccccaatg
ccacgctagcactgacggcgggcgtgctggtggactcggccgtggaggtggccttcctgtggacctttggggatggggagca
ggccctccaccagttccagcctccgtacaacgagtccttcccggttccagacccctcggtggcccaggtgctggtggagcaca
atgtcatgcacacctacgctgccccaggtgagtacctcctgaccgtgctggcatctaatgccttcgagaacctgacgcagcagg
tgcctgtgagcgtgcgcgcctccctgccctccgtggctgtgggtgtgagtgacggcgtcctggtggccggccggcccgtcacc
ttctacccgcacccgctgccctcgcctgggggtgttctttacacgtgggacttcggggacggctcccctgtcctgacccagagc
cagccggctgccaaccacacctatgcctcgaggggcacctaccacgtgcgcctggaggtcaacaacacggtgagcggtgcg
gcggcccaggcggatgtgcgcgtctttgaggagctccgcggactcagcgtggacatgagcctggccgtggagcagggcgc
ccccgtggtggtcagcgccgcggtgcagacgggcgacaacatcacgtggaccttgacatgggggacggcaccgtgctgtc
gggcccggaggcaacagtggagcatgtgtacctgcgggcacagaactgcacagtgaccgtgggtcggccagccccgccg
gccacctggcccggagcctgcacgtgctggtcttcgtcctggaggtgctgcgcgttgaacccgccgcctgcatccccacgcag
cctgacgcgcggctcacggcctacgtcaccgggaacccggcccactacctcttcgactggaccttcggggatggctcctccaa
cacgaccgtgcgggggtgcccgacggtgacacacaacttcacgcggagcggcacgttccccctggcgctggtgctgtccag
ccgcgtgaacagggcgcattacttcaccagcatctgcgtggagccagaggtgggcaacgtcaccctgcagccagagaggca
gtttgtgcagctcggggacgaggcctggctggtggcatgtgcctggcccccgttcccctaccgctacacctgggactttggcac
cgaggaagccgcccccacccgtgccaggggccctgaggtgacgttcatctaccgagacccaggctcctatcttgtgacagtca
ccgcgtccaacaacatctctgctgccaatgactcagccctggtggaggtgcaggagcccgtgctggtcaccagcatcaaggtc
aatggctcccttgggctggagctgcagcagccgtacctgttctctgctgtgggccgtgggcgccccgccagctacctgtgggat
ctgggggacggtgggtggctcgagggtccggaggtcacccacgcttacaacagcacaggtgacttcaccgttagggtggcc
ggctggaatgaggtgagccgcagcgaggcctggctcaatgtgacggtgaagcggcgcgtgcgggggctcgtcgtcaatgca
agccgcacggtggtgcccctgaatgggagcgtgagcttcagcacgtcgctggaggccggcagtgatgtgcgctattcctgggt
gctctgtgaccgctgcacgcccatccctgggggtcctaccatctcttacaccttccgctccgtgggcaccttcaatatcatcgtca
cggctgagaacgaggtgggctccgcccaggacagcatcttcgtctatgtcctgcagctcatagaggggctgcaggtggtggg
cggtggccgctacttccccaccaaccacacggtacagctgcaggccgtggttagggatggcaccaacgtctcctacagctgga
ctgcctggagggacaggggcccggccctggccggcagcggcaaaggcttctcgctcaccgtgctcgaggccggcacctac
catgtgcagctgcgggccaccaacatgctgggcagcgcctgggccgactgcaccatggacttcgtggagcctgtggggtggc
tgatggtggccgcctccccgaacccagctgccgtcaacacaagcgtcaccctcagtgccgagctggctggtggcagtggtgtc
gtatacacttggtccttggaggaggggctgagctgggagacctccgagccatttaccacccatagcttccccacacccggcctg
cacttggtcaccatgacggcagggaacccgctgggctcagccaacgccaccgtggaagtggatgtgcaggtgcctgtgagtg
gcctcagcatcagggccagcgagcccggacaccatcaccatcaccatcaccatcaccattag
Protein sequence of PKD1 extracellular fragment domains PKDI-PKDX)
expressed in Pichia Pastoris using pPIC9 expression vector (Life Technologies). His tag
used for purification is underlined. (SEQ ID NO: 15)
(SEQ ID NO: 15)
ATRWDFGDGSAEVDAAGPAASHRYVLPGRYHVTAVLALGAGSALLGTD
VQVEAAPAALELVCPSSVQSDESLDLSIQNRGGSGLEAAYSIVALGEEPARAVHP
LCPSDTEIFPGNGHCYRLVVEKAAWLQAQEQCQAWAGAALAMVDSPAVQRFL
VSRVTRSLDVWIGESTVQGVEVGPAPQGEAFSLESCQNWLPGEPHPATAEHCVR
LGPTGWCNTDLCSAPHSYVCELQPGGPVQDAENLLNGAPSGDLQGPLTPLAQQ
DGLSAPHEPVEVMVFPGLRLSREAFLTTAEFGTQELRRPAQLRLQVYRLLSTAGT
PENGSEPESRSPDNRTQLAPACMPGGRWCTGANICLPLDASCHPQACANGCTSG
PGLPGAPYALWREFLFSVPAGPPAQYSVTLHGQDVLMLPGDLVGLQHDAGPGA
LLHCSPAPGHPGPQAPYLSANASSWLPHLPAQLEGTWACPACALRLLAATEQLT
VLLGLRPNPGLRLPGRYEVRAEVGNGVSRHNLSCSFDVVSPVAGLRVIYPAPRD
GRLYVPTNGSALVLQVDSGANATATARWPGGSVSARFENVCPALVATFVPGCP
WETNDTLFSVVALPWLSEGEHVVDVVVENSASRANLSLRVTAEEPICGLRATPS
PEARVLQGVLNRYSTVVEAGSDMVFRWTINDKQSLTFQNVVFNVIYQSAAVFK
LSLTASNHVSNVTVNYNVTVERMNRMQGLQVSTVPAVLSPNATLALTAGVLVD
SAVEVAFLWTFGDGEQALHQFQPPYNESFPVPDPSVAQVLVEHNVMHTYAAPG
EYLLTVLASNAFENLTQQVPVSVRASLPSVAVGVSDGVLVAGRPVTFYPHPLPSP
GGVLYTWDFGDGSPVLTQSQPAANHTYASRGTYHVRLEVNNTVSGAAAQADV
RVFEELRGLSVDMSLAVEQGAPVVVSAAVQTGDNITWTFDMGDGTVLSGPEAT
VEHVYLRAQNCTVTVGAASPAGHLARSLHVLVFVLEVLRVEPAACIPTQPDARL
TAYVTGNPAHYLFDWTFGDGSSNTTVRGCPTVTHNFTRSGTFPLALVLSSRVNR
AHYFTSICVEPEVGNVTLQPERQFVQLGDEAWLVACAWPPFPYRYTWDFGTEE
AAPTRARGPEVTFIYRDPGSYLVTVTASNNISAANDSALVEVQEPVLVTSIKVNG
SLGLELQQPYLFSAVGRGRPASYLWDLGDGGWLEGPEVTHAYNSTGDFTVRVA
GWNEVSRSEAWLNVTVKRRVRGLVVNASRTVVPLNGSVSFSTSLEAGSDVRYS
WVLCDRCTPIPGGPTISYTFRSVGTFNIIVTAENEVGSAQDSIFVYVLQLIEGLQV
VGGGRYFPTNHTVQLQAVVRDGTNVSYSWTAWRDRGPALAGSGKGFSLTVLE
AGTYHVQLRATNMLGSAWADCTMDFVEPVGWLMVAASPNPAAVNTSVTLSA
ELAGGSGVVYTWSLEEGLSWETSEPFTTHSFPTPGLHLWMTAGNPLGSANATV
EVDVQVPVSGLSIRASEPGHHHHHHHHHH.
DNA sequence of PKD1 extracellular fragment (PKD domain XII and REJ).
DNA sequence was cloned in to expression vector pPIC9 (Life Technologies) for
expression and purification in Pichia Pastoris. His tag used for purification is
underlined. (SEQ ID NO: 16)
(SEQ ID NO: 16)
aaccgctcggcgcagtttgaggccgccaccagccccagcccccggcgtgtggcctaccactgggactttgggat
gggtcgccagggcaggacacagatgagcccagggccgagcactcctacctgaggcctgggaactaccgcgtgcaggtgaa
cgcctccaacctggtgagcttcttcgtggcgcaggccacggtgaccgtccaggtgctggcctgccgggagccggaggtggac
gtggtcctgcccctgcaggtgctgatgcggcgatcacagcgcaactacttggaggcccacgttgacctgcgcgactgcgtcac
ctaccagactgagtaccgctgggaggtgtatcgcaccgccagctgccagcggccggggcgcccagcgcgtgtggccctgcc
cggcgtggacgtgagccggcctcggctggtgctgccgcggctggcgctgcctgtggggcactactgctttgtgtttgtcgtgtc
atttggggacacgccactgacacagagcatccaggccaatgtgacggtggcccccgagcgcctggtgcccatcattgagggt
ggctcataccgcgtgtggtcagacacacgggacctggtgctggatgggagcgagtcctacgaccccaacctggaggacggc
gaccagacgccgctcagtttccactgggcctgtgtggcttcgacacagagggaggctggcgggtgtgcgctgaactttgggcc
ccgcgggagcagcacggtcaccattccacgggagcggctggcggctggcgtggagtacaccttcagcctgaccgtgtggaa
ggccggccgcaaggaggaggccaccaaccagacggtgctgatccggagtggccgggtgcccattgtgtccttggagtgtgt
gtcctgcaaggcacaggccgtgtacgaagtgagccgcagctcctacgtgtacttggagggccgctgcctcaattgcagcagc
ggctccaagcgagggcggtgggctgcacgtacgttcagcaacaagacgctggtgctggatgagaccaccacatccacgggc
agtgcaggcatgcgactggtgctgcggcggggcgtgctgcgggacggcgagggatacaccttcacgctcacggtgctgggc
cgctctggcgaggaggagggctgcgcctccatccgcctgtcccccaaccgcccgccgctggggggctcttgccgcctcttcc
cactgggcgctgtgcacgccctcaccaccaaggtgcacttcgaatgcacgggctggcatgacgcggaggatactggcgccc
cgctggtgtacgccctgctgctgcggcgctgtcgccagggccactgcgaggagttctgtgtctacaagggcagcctctccagc
tacggagccgtgctgcccccgggtttcaggccacacttcgaggtgggcctggccgtggtggtgcaggaccagctgggagcc
gctgtggtcgccctcaacaggtctttggccatcaccctcccagagcccaacggcagcgcaacggggctcacagtctggctgca
cgggctcaccgctagtgtgctcccagggctgctgcggcaggccgatccccagcacgtcatcgagtactcgttggccctggtca
ccgtgctgaacgagtacgagcgggccctggacgtggcggcagagcccaagcacgagcggcagcaccgagcccagatacg
caagaacatcacggagactctggtgtccctgagggtccacactgtggatgacatccagcagatcgctgctgcgctggcccagt
gcatggggcccagcagggagctcgtatgccgctcgtgcctgaagcagacgctgcacaagctggaggccatgatgctcatcct
gcaggcagagaccaccgcgggcaccgtgacgcccaccgccatcggagacagcatcctcaacatcacaggagacctcatcc
acctggccagctcggacgtgcgggcaccacagccctcagagctgggagccgagtcaccatctcggatggtggcgtcccagg
cctacaacctgacctctgccctcatgcgcatcctcatgcgctcccgcgtgctcaacgaggagcccctgacgcggcgggcgag
gagatcgtggcccagggcaagcgctcggacccgcggagcctgctgtgctatggcggcgccccagggcctggctgccacttc
tccatccccgaggctttcagcggggccctggccaacctcagtgacgtggtgcagctcatctttctggtggactccaatccctttcc
ctttggctatatcagcaactacaccgtctccaccaaggtgcaccatcaccatcaccatcaccatcaccattag
Protein sequence of PKD1 extracellular fragment (PKD domain XII and REJ)
expressed in Pichia Pastoris using pPIC9 expression vector (Life Technologies). His tag
used for purification is underlined. (SEQ ID NO: 17)
(SEQ ID NO: 17)
NRSAQFEAATSPSPRRVAYHWDFGDGSPGQDTDEPRAEHSYLRPGDYRV
QVNASNLVSFFVAQATVTVQVLACREPEVDVVLPLQVLMRRSQRNYLEAHVDL
RDCVTYQTEYRWEVYRTASCQRPGRPARVALPGVDVSRPRLVLPRLALPVGHY
CFVFVVSFGDTPLTQSIQANVTVAPERLVPIIEGGSYRVWSDTRDLVLDGSESYD
PNLEDGDQTPLSFHWACVASTQREAGGCALNFGPRGSSTVTIPRERLAAGVEYT
FSLTVWKAGRKEEATNQTVLIRSGRVPIVSLECVSCKAQAVYEVSRSSYVYLEG
RCLNCSSGSKRGRWAARTFSNKTLVLDETTTSTGSAGMRLVLRRGVLRDGEGY
TFTLTVLGRSGEEEGCASIRLSPNRPPLGGSCRLFPLGAVHALTTKVHFECTGWH
DAEDAGAPLVYALLLRRCRQGHCEEFCVYKGSLSSYGAVLPPGFRPHFEVGLAV
VVQDQIGAAVVALNRSLAITLPEPNGSATGLTVWLHGLTASVLPGLLRQADPQ
HVIEYSLALVTVLNEYERALDVAAEPKHERQHRAQIRKNITETLVSLRVHTVDDI
QQIAAALAQCMGPSRELVCRSCLKQTLHKLEAMMLILQAETTAGTVTPTAIGDSI
LNITGDLIHLASSDVRAPQPSELGAESPSRMVASQAYNLTSALMRILMRSRVLNE
EPLTLAGEEIVAQGKRSDPRSLLCYGGAPGPGCHFSIPEAFSGALANLSDVVQLIF
LVDSNPFPEGYISNYTVSTKVHHHHHHHHHH.
DNA sequence of PKD1L1 extracellular fragment. DNA sequence was cloned
into expression vector pPIC9 (Life Technologies) for expression and purification in
Pichia Pastoris. His tag used for purification is underlined. (SEQ ID NO: 18)
(SEQ ID NO: 18)
atggccgaggaggcagcccagaacatttctgatgaccaggaaaggtgtctccaggctgcctgctgcctttcctttggt
ggtgagctgtctgtgagcactgacaagagaggggtcttcatctgtgcagctgtagccctcctggaggtggattgtgggtcgag
gtctatgctaatcatgtgcttcttatgagtgatgggaagtgtggctgtccttggtgtgctctgaatggaaaggcagaagaccggga
atcacagagcccatcctcatcagcttccaggcagaagaacatttggaaaacaactagcgaagcagcgttaagtgttgttaatgaa
aaaacacaggctgttgttaatgaaaaaacacaggcgcctggattgtgataacagtgctgatagaattccccacaaacctttcat
tataatcgcaagggcctggagcagtggtggccccaggttccatcacaggcggctgtgtgctactgggaccgcagacagcaca
ttctgctgctcttctccagctccagggcaccacttctgcagcagctccctgcagcctgaagatggaggcttcctgctgtgtcctgag
actgctgtgctgtgcggaggatgtggccacggggctgcttcctgggactgtcacgatggagacccccaccaaggtggccaga
cccactcagaccagtcccagcgagtgcccctgtggccgatttcacattttcccacttctcccagaagctcccacggccttccgc
ctggcattcctcgcacccccagcttcacggcatcgcagtctggttctgagatcctctatccccctactcagcatcctcctgtggcc
atcctagctcgaaattctgataacttcatgaaccctgttcttaattgctccctggaagtggaagctcgggcacctccaaatctggga
ttccgtgttcatatggcttctggagaggctctctgtctgatgatggatttcggggacagttctggggttgaaatgaggctacacaac
atgtctgaggcaatggcggtgactgcctaccaccagtactcaaaaggtattttttttcatcttttacattttcagttgatatgtccacc
tacaaagaagcagagacacaaaatacaactttaaatgtttacttgtgccaaagtgaaaacagctgcctggaagactcagacccc
agtaaccttggatatgagcttatttctgcctttgtcaccaaaggagtcatatgctcaaggctgttatttataacgagtttcatggaacc
gaagtggagcttgggccttattatgtggagattggccatgaggccgtgtctgcgttcatgaactccagcagtgtccatgaagatg
aagtgcttgtctttgctgactcccaagtgaatcagaaaagcactgtggttatacatcactttccatctattccttcatataacgtgtcct
ttatttctcagactcaagtgggtgacagccaggcttggcacagcatgactgtctggtataagatgcaatccgtctctgtctacacaa
atggaactgtgtttgccacagacacagacattacatttacagctgttaccaaggaaacaatacccctggaatttgagtggtattttg
gagaggacccaccagtgaggacaacttcaagaagcattaaaaaaagactcagcatcccccaatggtatcgtgtgatggttaag
gcttccaacaggatgagcagtgtggtctctgagccccatgtcatcagggtgcagaagaaaattgtggccaatcggctccaccat
caccatcaccatcaccatcaccattag
Protein sequence of PKD1 extracellular fragment (domains PKDI-PKDX)
expressed in Pichia Pastoris using pPIC9 expression vector (Life Technologies). His tag
used for purification is underlined. (SEQ ID NO: 19)
(SEQ ID NO: 19)
MAEEAAQNISDDQERCLQAACCLSFGGELSVSTDKSWGLHLCSCSPPGG
GLWVEVYANHVLLMSDGKCGCPWCALNGKAEDRESQSPSSSASRQKNIWKTTS
EAALSVVNEKTQAVVNEKTQAPLDCDNSADRIPIIKPFIIIARAWSSGGPRFHHRR
LCATGTADSTFSALLQLQGTTSAAAPCSLKMEASCCVLRLLCCAEDVATGLLPG
TVTMETPTKVARPTQTSSQRVPLWPISHFPTSPRSSHGLPPGIPRTPSFTASQSGSEI
LYPPTQHPPVAILARNSDNFMNPVLNCSLEVEARAPPNLGFRVHMASGEALCLM
MDFGDSSGVEMRLHNMSEAMAVTAYHQYSKGIFFHLLHFQLDMSTYKEAETQ
NTTLNVYLCQSENSCLEDSDPSNLGYELISAFVTKGVYMLKAVIYNEFHGTEVEL
GPYYVEIGHEAVSAFMNSSSVHEDEVLVEADSQVNQKSTVVIHHFPSIPSYNVSFI
SQTQVGDSQAWHSMTVWYKMQSVSVYTNGTVFATDTDITFTAVTKETIPLEFE
WYFGEDPPVRTTSRSIKKRLSIPQWYRVMVKASNRMSSVVSEPHVIRVQKKIVA
NRLHHHHHHHHHH.

All references cited herein are fully incorporated by reference. Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

1. A method for identifying a compound that modulates polycystin-1/polycystin-2 (PKD1/PKD2) activity, comprising:
contacting a cell having a plasma membrane PKD1/PKD2 with a test compound, detecting whether PKD1/PKD2 activity is modulated in the presence of the test compound with respect to PKD1/PKD2 activity in the absence of the test compound, and wherein if the PKD1/PKD2 activity is modulated then the test compound is a compound that modulates PKD1/PKD2 activity.

2. The method of claim 1, wherein the method of detection comprises a voltage-clamp, patch clamp, x-ray crystallization, electron microscopy, circular dichroism, Fourier transform infra-red spectroscopy, electron spin resonance, nuclear magnetic resonance spectroscopy, flow cytometry, immunodetection fluorescence techniques, surface biotinylation, calcium imaging techniques, or atomic force microscopy.

3. The method of claim 1, wherein the test compound comprises a small molecule, peptide, nucleic acid or polysaccharide including, but not limited to antibodies and biologics.

4. The method of claim 1, wherein the test compound comprises an inhibitor of PKD1/PKD2 activity.

5. The method of claim 1, wherein the test compound comprises an activator of PKD1/PKD2 activity.

6. The method of claim 1, wherein the test compound comprises a trafficking modulator to the plasma membrane or primary cilium.

7. The method of claim 1, wherein the PKD1 or PKD2 is a chimera.

8. The method of claim 1, wherein the PKD1 or PKD2 includes an intracellular or extracellular tag.

9. The method of claim 1, wherein N-terminal truncations of PKD1 enhance PKD2 surface trafficking.

10. The method of claim 1, wherein C-terminal truncations of PKD1 enhance PKD2 surface trafficking.

11. (canceled)

12. The method of claim 1, wherein PKD1 in the plasma membrane PKD1/PKD2 comprises a modified PKD1.

13. The method of claim 12, wherein the modified PKD1 is a P2Y12-PKD1 P2Y12-PKD1L1, P2Y12-PKD1L2 or P2Y2-PKD1L3.

14. The method of claim 12, wherein the N-terminus of the modified PKD1 does not contain P2Y12.

15. The method of claim 12, wherein the modified PKD1/PKD2 complex contains autosomal dominant polycystic kidney disease (ADPKD) causing mutations in either PKD1 or PKD2 which affects plasma membrane trafficking and/or alters PKD1/PKD2 ion channel function.

16. The method of claim 12, wherein the modified PKD1/PKD2 complex contains ADPKD causing mutations in PKD1 which affects plasma membrane trafficking and/or alters PKD1/PKD2 ion channel function.

17. The method of claim 12, wherein the modified PKD1/PKD2 complex contains ADPKD disease causing mutations in either PKD1 or PKD2 which affects or alters PKD1/PKD2 ion channel function.

18. The method of claim 1, wherein the PKD1 comprises SEQ ID NO: 3, SEQ ID NO: 4, SEQ ID NO: 5, SEQ ID NO: 6, SEQ ID NO: 7, or SEQ ID NO: 8.

19. The method of claim 1, wherein the PKD2 comprises SEQ ID NO: 1, SEQ ID NO: 2 or SEQ ID NO: 11.

20. A cell having a PKD1/PKD2 in a plasma membrane.

21. The cell of claim 20, wherein the PKD1 is a chimera.

22. The cell of claim 20, wherein the PKD2 is a chimera.

23. The cell of claim 20, wherein the PKD1 includes an intracellular or extracellular tag.

24. The cell of claim 20, wherein the PKD2 includes an intracellular or extracellular tag.

25-31. (canceled)

32. A chimeric PKD1 comprising a C-terminal PKD1 fragment linked to an N-terminal plasma membrane insertion domain.

33. The chimeric PKD1 of claim 32, wherein the plasma membrane insertion domain is P2Y12.

34. The chimeric PKD1 of claim 32, wherein the plasma membrane insertion domain is not P2Y12.

35. The chimeric PKD1 of claim 32, wherein the PKD1 includes an intracellular or extracellular tag.

36. The chimeric PKD1 of claim 35, wherein the tag is selected from the group consisting of a HA tag, His-tag, GFP, YFP, BirA, mCherry, ires GFP, ires mCherry, FLAG tag, and covalent labeling of fusion proteins using SNAP-, CLIP-, ACP- and MCP-tags.

37. The chimeric PKD1 of claim 32, wherein the C-terminal PKD1 fragment comprises SEQ ID NO: 3, SEQ ID NO: 4, SEQ ID NO: 5, SEQ ID NO: 6, SEQ ID NO: 7, or SEQ ID NO: 8.

38. The chimeric PKD1 of claim 32, wherein the N-terminal plasma membrane insertion domain comprises SEQ ID NO: 9 and SEQ ID NO: 10.

39-72. (canceled)


SEQUENCE LISTING (48 sequences)

SEQ ID NO: 1 - DNA, 2916 bp, Artificial Sequence
  Synthetic Polynucleotide
atggtgaactccagtcgcgtgcagcctcagcagcccggggacgccaagcggccgcccgcgccccgcgcgccggacccgggccggctgatggctggctgcgcggccgtgggcgccagcctcgccgccccgggcggcctctgcgagcagcggggcctggagatcgagatgcagcgcatccggcaggcggccgcgcgggaccccccggccggagccgcggcctccccttctcctccgctctcgtcgtgctcccggcaggcgtggagccgcgataaccccggcttcgaggccgaggaggaggaggaggaggtggaaggggaagaaggcggaatggtggtggagatggacgtagagtggcgcccgggcagccggaggtcggccgcctcctcggccgtgagctccgtgggcgcgcggagccgggggcttgggggctaccacggcgcgggccacccgagcgggaggcggcgccggcgagaggaccagggcccgccgtgccccagcccagtcggcggcggggacccgctgcatcgccacctccccctggaagggcagccgccccgagtggcctgggcggagaggctggttcgcgggctgcgaggtctctggggaacaagactcatggaggaaagcagcactaaccgagagaaataccttaaaagtgttttacgggaactggtcacatacctcctttttctcatagtcttgtgcatcttgacctacggcatgatgagctccaatgtgtactactacacccggatgatgtcacagctcttcctagacacccccgtgtccaaaacggagaaaactaactttaaaactctgtcttccatggaagacttctggaagttcacagaaggctccttattggatgggctgtactggaagatgcagcccagcaaccagactgactacaaagacgatgacgacaagagtttcatcttctatgagaacctgctgttaggggttccacgaatacggcaactccgagtcagaaatggatcctgctctatcccccaggacttgagagatgaaattaaagagtgctatgatgtctactctgtcagtagtgaagatagggctccctttgggccccgaaatggaaccgcttggatctacacaagtgaaaaagacttgaatggtagtagccactggggaatcattgcaacttatagtggagctggctattatctggatttgtcaagaacaagagaggaaacagctgcacaagttgctagcctcaagaaaaatgtctggctggaccgaggaaccagggcaacttttattgacttctcagtgtacaacgccaacattaacctgttctgtgtggtcaggttattggttgaattcccagcaacaggtggtgtgattccatcttggcaatttcagcctttaaagctgatccgatatgtcacaacttttgatttcttcctggcagcctgtgagattatcttttgtttctttatcttttactatgtggtggaagagatattggaaattcgcattcacaaactacactatttcaggagtttctggaattgtctggatgttgtgatcgttgtgctgtcagtggtagctataggaattaacatatacagaacatcaaatgtggaggtgctactacagtttctggaagatcaaaatactttccccaactttgagcatctggcatattggcagatacagttcaacaatatagctgctgtcacagtattttttgtctggattaagctcttcaaattcatcaattttaacaggaccatgagccagctctcgacaaccatgtctcgatgtgccaaagacctgtttggctttgctattatgttcttcattattttcctagcgtatgctcagttggcataccttgtctttggcactcaggtcgatgacttcagtactttccaagagtgtatcttcactcaattccgtatcattttgggcgatatcaactttgcagagattgaggaagctaatcgagttttgggaccaatttatttcactacatttgtgttctttatgttcttcattcttttgaatatgtttttggctatcatcaatgatacttactctgaagtgaaatctgacttggcacagcagaaagctgaaatggaactctcagatcttatcagaaagggctaccataaagctttggtcaaactaaaactgaaaaaaaataccgtggatgacatttcagagagtctgcggcaaggaggaggcaagttaaactttgacgaacttcgacaagatctcaaagggaagggccatactgatgcagagattgaggcaatattcacaaagtacgaccaagatggagaccaagaactgaccgaacatgaacatcagcagatgagagacgacttggagaaagagagggaggacctggatttggatcacagttctttaccacgtcccatgagcagccgaagtttccctcgaagcctggatgactctgaggaggatgacgatgaagatagcggacatagctccagaaggaggggaagcatttctagtggcgtttcttacgaagagtttcaagtcctggtgagacgagtggaccggatggagcattccatcggcagcatagtgtccaagattgacgccgtgatcgtgaagctagagattatggagcgagccaaactgaagaggagggaggtgctgggaaggctgttggatggggtggccgaggatgaaaggctgggtcgtgacagtgaaatccatagggaacagatggaacggctagtacgtgaagagttggaacgctgggaatccgatgatgcagcttcccagatcagtcatggtttaggcacgccagtgggactaaatggtcaacctcgccccagaagctcccgcccatcttcctcccaatctacagaaggcatggaaggtgcaggtggaaatgggagttctaatgtccacgtatga

SEQ ID NO: 2 - protein, 971 aa, Artificial Sequence
  Synthetic Polypeptide
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTDYKDDDDKSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLSVVAIGINIYRTSNVEVLLQFLEDQNTFPNFEHLAYWQIQFNNIAAVTVFFVWIKLFKFINFNRTMSQLSTTMSRCAKDLFGFAIMFFIIFLAYAQLAYLVFGTQVDDFSTFQECIFTQFRIILGDINFAEIEEANRVLGPIYFTTFVFFMFFILLNMFLAIINDTYSEVKSDLAQQKAEMELSDLIRKGYHKALVKLKLKKNTVDDISESLRQGGGKLNFDELRQDLKGKGHTDAEIEAIFTKYDQDGDQELTEHEHQQMRDDLEKEREDLDLDHSSLPRPMSSRSFPRSLDDSEEDDDEDSGHSSRRRGSISSGVSYEEFQVLVRRVDRMEHSIGSIVSKIDAVIVKLEIMERAKLKRREVLGRLLDGVAEDERLGRDSEIHREQMERLVREELERWESDDAASQISHGLGTPVGLNGQPRPRSSRPSSSQSTEGMEGAGGNGSSNVHV

SEQ ID NO: 3 - DNA, 3870 bp, Artificial Sequence
  Synthetic Polynucleotide
atgtacccatacgatgttccagattacgctcaagccgtcgacaacctcacctctgcgcctggtaacaccagtctgtgcaccagagactacaaaatcacccagaccgccttcggcgccagcctcttcgtgcccccaagccatgtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgagcaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttag

SEQ ID NO: 4 - protein, 1289 aa, Artificial Sequence
  Synthetic Polypeptide
MYPYDVPDYAQAVDNLTSAPGNTSLCTRDYKITQTAFGASLFVPPSHVRFVFPEPTADVNYIVMLTCAVCLVTYMVMAAILHKLDQLDASRGRAIPFCGQRGRFKYEILVKTGWGRGSGTTAHVGIMLYGVDSRSGHRHLDGDRAFHRNSLDIFRIATPHSLGSVWKIRVWHDNKGLSPAWFLQHVIVRDLQTARSAFFLVNDWLSVETEANGGLVEKEVLAASDAALLRFRRLLVAELQRGFFDKHIWLSIWDRPPRSRFTRIQRATCCVLLICLFLGANAVWYGAVGDSAYSTGHVSRLSPLSVDTVAVGLVSSVVVYPVYLAILFLFRMSRSKVAGSPSPTPAGQQVLDIDSCLDSSVLDSSFLTFSGLHAEQAFVGQMKSDLFLDDSKSLVCWPSGEGTLSWPDLLSDPSIVGSNLRQLARGQAGHGLGPEEDGFSLASPYSPAKSFSASDEDLIQQVLAEGVSSPAPTQDTHMETDLLSSLSSTPGEKTETLALQRLGELGPPSPGLNWEQPQAARLSRTGLVEGLRKRLLPAWCASLAHGLSLLLVAVAVAVSGWVGASFPPGVSVAWLLSSSASFLASFLGWEPLKVLLEALYFSLVAKRLHPDEDDTLVESPAVTPVSARVPRVRPPHGFALFLAKEEARKVKRLHGMLRSLLVYMLFLLVTLLASYGDASCHGHAYRLQSAIKQELHSRAFLAITRSEELWPWMAHVLLPYVHGNQSSPELGPPRLRQVRLQEALYPDPPGPRVHTCSAAGGFSTSDYDVGWESPHNGSGTWAYSAPDLLGAWSWGSCAVYDSGGYVQELGLSLEESRDRLRFLQLHNWLDNRSRAVFLELTRYSPAVGLHAAVTLRLEFPAAGRALAALSVRPFALRRLSAGLSLPLLTSVCLLLFAVHFAVAEARTWHREGRWRVLRLGAWARWLLVALTAATALVRLAQLGAADRQWTRFVRGRPRRFTSFDQVAQLSSAARGLAASLLFLLLVKAAQQLRFVRQWSVFGKTLCRALPELLGVTLGLVVLGVAYAQLAILLVSSCVDSLWSVAQALLVLCPGTGLSTLCPAESWHLSPLLCVGLWALRLWGALRLGAVILRWRYHALRGELYRPAWEPQDYEMVELFLRRLRLWMGLSKVKEFRHKVRFEGMEPLPSRSSRGSKVSPDVPPPSAGSDASHPSTSSSQLDGLSVSLGRLGTRCEPEPSRLQAVFEALLTQFDRLNQATEDVYQLEQQLHSLQGRRSSRAPAGSSRGPSPGLRPALPSRLARASRGVDLATGPSRTPLRAKNKVHPSST

SEQ ID NO: 5 - DNA, 3831 bp, Artificial Sequence
  Synthetic Polynucleotide
atgtacccatacgatgttccagattacgctcaagccgtcgacaacctcacctctgcgcctggtaacaccagtctgtgcaccagagactacaaaatcacccaggtccgctttgtgtttcctgagccgacagcggatgtaaactacatcgtcatgctgacatgtgctgtgtgcctggtgacctacatggtcatggccgccatcctgcacaagctggaccagttggatgccagccggggccgcgccatccctttctgtgggcagcggggccgcttcaagtacgagatcctcgtcaagacaggctggggccggggctcaggtaccacggcccacgtgggcatcatgctgtatggggtggacagccggagcggccaccggcacctggacggcgacagagccttccaccgcaacagcctggacatcttccggatcgccaccccgcacagcctgggtagcgtgtggaagatccgagtgtggcacgacaacaaagggctcagccctgcctggttcctgcagcacgtcatcgtcagggacctgcagacggcacgcagcgccttcttcctggtcaatgactggctttcggtggagacggaggccaacgggggcctggtggagaaggaggtgctggccgcgagcgacgcagcccttttgcgcttccggcgcctgctggtggctgagctgcagcgtggcttctttgacaagcacatctggctctccatatgggaccggccgcctcgtagccgtttcactcgcatccagagggccacctgctgcgttctcctcatctgcctcttcctgggcgccaacgccgtgtggtacggggctgttggcgactctgcctacagcacggggcatgtgtccaggctgagcccgctgagcgtcgacacagtcgctgttggcctggtgtccagcgtggttgtctatcccgtctacctggccatcctttttctcttccggatgtcccggagcaaggtggctgggagcccgagccccacacctgccgggcagcaggtgctggacatcgacagctgcctggactcgtccgtgctggacagctccttcctcacgttctcaggcctccacgctgagcaggcctttgttggacagatgaagagtgacttgtttctggatgattctaagagtctggtgtgctggccctccggcgagggaacgctcagttggccggacctgctcagtgacccgtccattgtgggtagcaatctgcggcagctggcacggggccaggcgggccatgggctgggcccagaggaggacggcttctccctggccagcccctactcgcctgccaaatccttctcagcatcagatgaagacctgatccagcaggtccttgccgagggggtcagcagcccagcccctacccaagacacccacatggaaacggacctgctcagcagcctgtccagcactcctggggagaagacagagacgctggcgctgcagaggctgggggagctggggccacccagcccaggcctgaactgggaacagccccaggcagcgaggctgtccaggacaggactggtggagggtctgcggaagcgcctgctgccggcctggtgtgcctccctggcccacgggctcagcctgctcctggtggctgtggctgtggctgtctcagggtgggtgggtgcgagcttccccccgggcgtgagtgttgcgtggctcctgtccagcagcgccagcttcctggcctcattcctcggctgggagccactgaaggtcttgctggaagccctgtacttctcactggtggccaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttag

SEQ ID NO: 6 - protein, 1276 aa, Artificial Sequence
  Synthetic Polypeptide
MYPYDVPDYAQAVDNLTSAPGNTSLCTRDYKITQVRFVFPEPTADVNYIVMLTCAVCLVTYMVMAAILHKLDQLDASRGRAIPFCGQRGRFKYEILVKTGWGRGSGTTAHVGIMLYGVDSRSGHRHLDGDRAFHRNSLDIFRIATPHSLGSVWKIRVWHDNKGLSPAWFLQHVIVRDLQTARSAFFLVNDWLSVETEANGGLVEKEVLAASDAALLRFRRLLVAELQRGFFDKHIWLSIWDRPPRSRFTRIQRATCCVLLICLFLGANAVWYGAVGDSAYSTGHVSRLSPLSVDTVAVGLVSSVVVYPVYLAILFLFRMSRSKVAGSPSPTPAGQQVLDIDSCLDSSVLDSSFLTFSGLHAEQAFVGQMKSDLFLDDSKSLVCWPSGEGTLSWPDLLSDPSIVGSNLRQLARGQAGHGLGPEEDGFSLASPYSPAKSFSASDEDLIQQVLAEGVSSPAPTQDTHMETDLLSSLSSTPGEKTETLALQRLGELGPPSPGLNWEQPQAARLSRTGLVEGLRKRLLPAWCASLAHGLSLLLVAVAVAVSGWVGASFPPGVSVAWLLSSSASFLASFLGWEPLKVLLEALYFSLVAKRLHPDEDDTLVESPAVTPVSARVPRVRPPHGFALFLAKEEARKVKRLHGMLRSLLVYMLFLLVTLLASYGDASCHGHAYRLQSAIKQELHSRAFLAITRSEELWPWMAHVLLPYVHGNQSSPELGPPRLRQVRLQEALYPDPPGPRVHTCSAAGGFSTSDYDVGWESPHNGSGTWAYSAPDLLGAWSWGSCAVYDSGGYVQELGLSLEESRDRLRFLQLHNWLDNRSRAVFLELTRYSPAVGLHAAVTLRLEFPAAGRALAALSVRPFALRRLSAGLSLPLLTSVCLLLFAVHFAVAEARTWHREGRWRVLRLGAWARWLLVALTAATALVRLAQLGAADRQWTRFVRGRPRRFTSFDQVAQLSSAARGLAASLLFLLLVKAAQQLRFVRQWSVFGKTLCRALPELLGVTLGLVVLGVAYAQLAILLVSSCVDSLWSVAQALLVLCPGTGLSTLCPAESWHLSPLLCVGLWALRLWGALRLGAVILRWRYHALRGELYRPAWEPQDYEMVELFLRRLRLWMGLSKVKEFRHKVRFEGMEPLPSRSSRGSKVSPDVPPPSAGSDASHPSTSSSQLDGLSVSLGRLGTRCEPEPSRLQAVFEALLTQFDRLNQATEDVYQLEQQLHSLQGRRSSRAPAGSSRGPSPGLRPALPSRLARASRGVDLATGPSRTPLRAKNKVHPSST

SEQ ID NO: 7 - DNA, 2058 bp, Homo sapiens
atgaagcggctgcacccggatgaagatgacaccctggtagagagcccggctgtgacgcctgtgagcgcacgtgtgccccgcgtacggccaccccacggctttgcactcttcctggccaaggaagaagcccgcaaggtcaagaggctacatggcatgctgcggagcctcctggtgtacatgctttttctgctggtgaccctgctggccagctatggggatgcctcatgccatgggcacgcctaccgtctgcaaagcgccatcaagcaggagctgcacagccgggccttcctggccatcacgcggtctgaggagctctggccatggatggcccacgtgctgctgccctacgtccacgggaaccagtccagcccagagctggggcccccacggctgcggcaggtgcggctgcaggaagcactctacccagaccctcccggccccagggtccacacgtgctcggccgcaggaggcttcagcaccagcgattacgacgttggctgggagagtcctcacaatggctcggggacgtgggcctattcagcgccggatctgctgggggcatggtcctggggctcctgtgccgtgtatgacagcgggggctacgtgcaggagctgggcctgagcctggaggagagccgcgaccggctgcgcttcctgcagctgcacaactggctggacaacaggagccgcgctgtgttcctggagctcacgcgctacagcccggccgtggggctgcacgccgccgtcacgctgcgcctcgagttcccggcggccggccgcgccctggccgccctcagcgtccgcccctttgcgctgcgccgcctcagcgcgggcctctcgctgcctctgctcacctcggtgtgcctgctgctgttcgccgtgcacttcgccgtggccgaggcccgtacttggcacagggaagggcgctggcgcgtgctgcggctcggagcctgggcgcggtggctgctggtggcgctgacggcggccacggcactggtacgcctcgcccagctgggtgccgctgaccgccagtggacccgtttcgtgcgcggccgcccgcgccgcttcactagcttcgaccaggtggcgcagctgagctccgcagcccgtggcctggcggcctcgctgctcttcctgcttttggtcaaggctgcccagcagctacgcttcgtgcgccagtggtccgtctttggcaagacattatgccgagctctgccagagctcctgggggtcaccttgggcctggtggtgctcggggtagcctacgcccagctggccatcctgctcgtgtcttcctgtgtggactccctctggagcgtggcccaggccctgttggtgctgtgccctgggactgggctctctaccctgtgtcctgccgagtcctggcacctgtcacccctgctgtgtgtggggctctgggcactgcggctgtggggcgccctacggctgggggctgttattctccgctggcgctaccacgccttgcgtggagagctgtaccggccggcctgggagccccaggactacgagatggtggagttgttcctgcgcaggctgcgcctctggatgggcctcagcaaggtcaaggagttccgccacaaagtccgctttgaagggatggagccgctgccctctcgctcctccaggggctccaaggtatccccggatgtgcccccacccagcgctggctccgatgcctcgcacccctccacctcctccagccagctggatgggctgagcgtgagcctgggccggctggggacaaggtgtgagcctgagccctcccgcctccaagccgtgttcgaggccctgctcacccagtttgaccgactcaaccaggccacagaggacgtctaccagctggagcagcagctgcacagcctgcaaggccgcaggagcagccgggcgcccgccggatcttcccgtggcccatccccgggcctgcggccagcactgcccagccgccttgcccgggccagtcggggtgtggacctggccactggccccagcaggacaccccttcgggccaagaacaaggtccaccccagcagcacttag

SEQ ID NO: 8 - protein, 685 aa, Homo sapiens
MKRLHPDEDDTLVESPAVTPVSARVPRVRPPHGFALFLAKEEARKVKRLHGMLRSLLVYMLFLLVTLLASYGDASCHGHAYRLQSAIKQELHSRAFLAITRSEELWPWMAHVLLPYVHGNQSSPELGPPRLRQVRLQEALYPDPPGPRVHTCSAAGGFSTSDYDVGWESPHNGSGTWAYSAPDLLGAWSWGSCAVYDSGGYVQELGLSLEESRDRLRFLQLHNWLDNRSRAVFLELTRYSPAVGLHAAVTLRLEFPAAGRALAALSVRPFALRRLSAGLSLPLLTSVCLLLFAVHFAVAEARTWHREGRWRVLRLGAWARWLLVALTAATALVRLAQLGAADRQWTRFVRGRPRRFTSFDQVAQLSSAARGLAASLLFLLLVKAAQQLRFVRQWSVFGKTLCRALPELLGVTLGLVVLGVAYAQLAILLVSSCVDSLWSVAQALLVLCPGTGLSTLCPAESWHLSPLLCVGLWALRLWGALRLGAVILRWRYHALRGELYRPAWEPQDYEMVELFLRRLRLWMGLSKVKEFRHKVRFEGMEPLPSRSSRGSKVSPDVPPPSAGSDASHPSTSSSQLDGLSVSLGRLGTRCEPEPSRLQAVFEALLTQFDRLNQATEDVYQLEQQLHSLQGRRSSRAPAGSSRGPSPGLRPALPSRLARASRGVDLATGPSRTPLRAKNKVHPSST

SEQ ID NO: 9 - DNA, 102 bp, Artificial Sequence
  Synthetic Polynucleotide
atgtacccatacgatgttccagattacgctcaagccgtcgacaacctcacctctgcgcctggtaacaccagtctgtgcaccagagactacaaaatcacccag

SEQ ID NO: 10 - protein, 34 aa, Artificial Sequence
  Synthetic Polypeptide
MYPYDVPDYAQAVDNLTSAPGNTSLCTRDYKITQ

SEQ ID NO: 11 - protein, 963 aa, Homo sapiens
MVNSSRVQPQQPGDAKRPPAPRAPDPGRLMAGCAAVGASLAAPGGLCEQRGLEIEMQRIRQAAARDPPAGAAASPSPPLSSCSRQAWSRDNPGFEAEEEEEEVEGEEGGMVVEMDVEWRPGSRRSAASSAVSSVGARSRGLGGYHGAGHPSGRRRRREDQGPPCPSPVGGGDPLHRHLPLEGQPPRVAWAERLVRGLRGLWGTRLMEESSTNREKYLKSVLRELVTYLLFLIVLCILTYGMMSSNVYYYTRMMSQLFLDTPVSKTEKTNFKTLSSMEDFWKFTEGSLLDGLYWKMQPSNQTSFIFYENLLLGVPRIRQLRVRNGSCSIPQDLRDEIKECYDVYSVSSEDRAPFGPRNGTAWIYTSEKDLNGSSHWGIIATYSGAGYYLDLSRTREETAAQVASLKKNVWLDRGTRATFIDFSVYNANINLFCVVRLLVEFPATGGVIPSWQFQPLKLIRYVTTFDFFLAACEIIFCFFIFYYVVEEILEIRIHKLHYFRSFWNCLDVVIVVLSVVAIGINIYRTSNVEVLLQFLEDQNTFPNFEHLAYWQIQFNNIAAVTVFFVWIKLFKFINFNRTMSQLSTTMSRCAKDLFGFAIMFFIIFLAYAQLAYLVFGTQVDDFSTFQECIFTQFRIILGDINFAEIEEANRVLGPIYFTTFVFFMFFILLNMFLAIINDTYSEVKSDLAQQKAEMELSDLIRKGYHKALVKLKLKKNTVDDISESLRQGGGKLNFDELRQDLKGKGHTDAEIEAIFTKYDQDGDQELTEHEHQQMRDDLEKEREDLDLDHSSLPRPMSSRSFPRSLDDSEEDDDEDSGHSSRRRGSISSGVSYEEFQVLVRRVDRMEHSIGSIVSKIDAVIVKLEIMERAKLKRREVLGRLLDGVAEDERLGRDSEIHREQMERLVREELERWESDDAASQISHGLGTPVGLNGQPRPRSSRPSSSQSTEGMEGAGGNGSSNVHV

SEQ ID NO: 12 - DNA, 2881 bp, Artificial Sequence
  Synthetic Polynucleotide
agcgatctgacggttcactaaacgagctctgcttatataggcctcccaccgtacacgccacctcgacatactcgagtttactccctatcagtgatagagaacgtatgaagagtttactccctatcagtgatagagaacgtatgcagactttactccctatcagtgatagagaacgtataaggagtttactccctatcagtgatagagaacgtatgaccagtttactccctatcagtgatagagaacgtatctacagtttactccctatcagtgatagagaacgtatatccagtttactccctatcagtgatagagaacgtataagctttaggcgtgtacggtgggcgcctataaaagcagagctcgtttagtgaaccgtcagatcgcctggagcaattccacaacacttttgtcttataccaactttccgtaccacttcctaccctcgtaaagtcgacaccggggcccagatctccgcggggatccatcgatccgcggccgccggcgatatctccagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgccccgagcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcggcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcctctagacatatgctgcagacgcgtggtacccccggggaattctccag

SEQ ID NO: 13 - DNA, 5297 bp, Artificial Sequence
  Synthetic Polynucleotide
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggactcagatctcgagctcaagcttcgaattctgcagtcgacggtaccgcggcgctacgtaaattccgccccccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctcycgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtatmagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcgrtrcacatgctttacatgtgtttagtcgaggttaaaaaaacgtctaggccccccgaaccacggggacgtggttttcctttgaaaaacacgatgataatatggccamaammawratggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

SEQ ID NO: 14 - DNA, 4569 bp, Artificial Sequence
  Synthetic Polynucleotide
gccacacgctgggacttcggagacggctccgccgaggtggatgccgctgggccggctgcctcgcatcgctatgtgctgcctgggcgctatcacgtgacggccgtgctggccctgggggccggctcagccctgctggggacagacgtgcaggtggaagcggcacctgccgccctggagctcgtgtgcccgtcctcggtgcagagtgacgagagccttgacctcagcatccagaaccgcggtggttcaggcctggaggccgcctacagcatcgtggccctgggcgaggagccggcccgagcggtgcacccgctctgcccctcggacacggagatcttccctggcaacgggcactgctaccgcctggtggtggagaaggcggcctggctgcaggcgcaggagcagtgtcaggcctgggccggggccgccctggcaatggtggacagtcccgccgtgcagcgcttcctggtctcccgggtcaccaggagcctagacgtgtggatcggcttctcgactgtgcagggggtggaggtgggcccagcgccgcagggcgaggccttcagcctggagagctgccagaactggctgcccggggagccacacccagccacagccgagcactgcgtccggctcgggcccaccgggtggtgtaacaccgacctgtgctcagcgccgcacagctacgtctgcgagctgcagcccggaggcccagtgcaggatgccgagaacctcctcgtgggagcgcccagtggggacctgcagggacccctgacgcctctggcacagcaggacggcctctcagccccgcacgagcccgtggaggtcatggtattcccgggcctgcgtctgagccgtgaagccttcctcaccacggccgaatttgggacccaggagctccggcggcccgcccagctgcggctgcaggtgtaccggctcctcagcacagcagggaccccggagaacggcagcgagcctgagagcaggtccccggacaacaggacccagctggcccccgcgtgcatgccagggggacgctggtgccctggagccaacatctgcttgccgctggacgcctcctgccacccccaggcctgcgccaatggctgcacgtcagggccagggctacccggggccccctatgcgctatggagagagttcctcttctccgttcccgcggggccccccgcgcagtactcggtcaccctccacggccaggatgtcctcatgctccctggtgacctcgttggcttgcagcacgacgctggccctggcgccctcctgcactgctcgccggctcccggccaccctggtccccaggccccgtacctctccgccaacgcctcgtcatggctgccccacttgccagcccagctggagggcacttgggcctgccctgcctgtgccctgcggctgcttgcagccacggaacagctcaccgtgctgctgggcttgaggcccaaccctggactgcggctgcctgggcgctatgaggtccgggcagaggtgggcaatggcgtgtccaggcacaacctctcctgcagctttgacgtggtctccccagtggctgggctgcgggtcatctaccctgccccccgcgacggccgcctctacgtgcccaccaacggctcagccttggtgctccaggtggactctggtgccaacgccacggccacggctcgctggcctgggggcagtgtcagcgcccgctttgagaatgtctgccctgccctggtggccaccttcgtgcccggctgcccctgggagaccaacgataccctgttctcagtggtagcactgccgtggctcagtgagggggagcacgtggtggacgtggtggtggaaaacagcgccagccgggccaacctcagcctgcgggtgacggcggaggagcccatctgtggcctccgcgccacgcccagccccgaggcccgtgtactgcagggagtcctagtgaggtacagccccgtggtggaggccggctcggacatggtcttccggtggaccatcaacgacaagcagtccctgaccttccagaacgtggtcttcaatgtcatttatcagagcgcggcggtcttcaagctctcactgacggcctccaaccacgtgagcaacgtcaccgtgaactacaacgtaaccgtggagcggatgaacaggatgcagggtctgcaggtctccacagtgccggccgtgctgtcccccaatgccacgctagcactgacggcgggcgtgctggtggactcggccgtggaggtggccttcctgtggacctttggggatggggagcaggccctccaccagttccagcctccgtacaacgagtccttcccggttccagacccctcggtggcccaggtgctggtggagcacaatgtcatgcacacctacgctgccccaggtgagtacctcctgaccgtgctggcatctaatgccttcgagaacctgacgcagcaggtgcctgtgagcgtgcgcgcctccctgccctccgtggctgtgggtgtgagtgacggcgtcctggtggccggccggcccgtcaccttctacccgcacccgctgccctcgcctgggggtgttctttacacgtgggacttcggggacggctcccctgtcctgacccagagccagccggctgccaaccacacctatgcctcgaggggcacctaccacgtgcgcctggaggtcaacaacacggtgagcggtgcggcggcccaggcggatgtgcgcgtctttgaggagctccgcggactcagcgtggacatgagcctggccgtggagcagggcgcccccgtggtggtcagcgccgcggtgcagacgggcgacaacatcacgtggaccttcgacatgggggacggcaccgtgctgtcgggcccggaggcaacagtggagcatgtgtacctgcgggcacagaactgcacagtgaccgtgggtgcggccagccccgccggccacctggcccggagcctgcacgtgctggtcttcgtcctggaggtgctgcgcgttgaacccgccgcctgcatccccacgcagcctgacgcgcggctcacggcctacgtcaccgggaacccggcccactacctcttcgactggaccttcggggatggctcctccaacacgaccgtgcgggggtgcccgacggtgacacacaacttcacgcggagcggcacgttccccctggcgctggtgctgtccagccgcgtgaacagggcgcattacttcaccagcatctgcgtggagccagaggtgggcaacgtcaccctgcagccagagaggcagtttgtgcagctcggggacgaggcctggctggtggcatgtgcctggcccccgttcccctaccgctacacctgggactttggcaccgaggaagccgcccccacccgtgccaggggccctgaggtgacgttcatctaccgagacccaggctcctatcttgtgacagtcaccgcgtccaacaacatctctgctgccaatgactcagccctggtggaggtgcaggagcccgtgctggtcaccagcatcaaggtcaatggctcccttgggctggagctgcagcagccgtacctgttctctgctgtgggccgtgggcgccccgccagctacctgtgggatctgggggacggtgggtggctcgagggtccggaggtcacccacgcttacaacagcacaggtgacttcaccgttagggtggccggctggaatgaggtgagccgcagcgaggcctggctcaatgtgacggtgaagcggcgcgtgcgggggctcgtcgtcaatgcaagccgcacggtggtgcccctgaatgggagcgtgagcttcagcacgtcgctggaggccggcagtgatgtgcgctattcctgggtgctctgtgaccgctgcacgcccatccctgggggtcctaccatctcttacaccttccgctccgtgggcaccttcaatatcatcgtcacggctgagaacgaggtgggctccgcccaggacagcatcttcgtctatgtcctgcagctcatagaggggctgcaggtggtgggcggtggccgctacttccccaccaaccacacggtacagctgcaggccgtggttagggatggcaccaacgtctcctacagctggactgcctggagggacaggggcccggccctggccggcagcggcaaaggcttctcgctcaccgtgctcgaggccggcacctaccatgtgcagctgcgggccaccaacatgctgggcagcgcctgggccgactgcaccatggacttcgtggagcctgtggggtggctgatggtggccgcctccccgaacccagctgccgtcaacacaagcgtcaccctcagtgccgagctggctggtggcagtggtgtcgtatacacttggtccttggaggaggggctgagctgggagacctccgagccatttaccacccatagcttccccacacccggcctgcacttggtcaccatgacggcagggaacccgctgggctcagccaacgccaccgtggaagtggatgtgcaggtgcctgtgagtggcctcagcatcagggccagcgagcccggacaccatcaccatcaccatcaccatcaccattag

SEQ ID NO: 15 - protein, 1522 aa, Artificial Sequence
  Synthetic Polypeptide
ATRWDFGDGSAEVDAAGPAASHRYVLPGRYHVTAVLALGAGSALLGTDVQVEAAPAALELVCPSSVQSDESLDLSIQNRGGSGLEAAYSIVALGEEPARAVHPLCPSDTEIFPGNGHCYRLVVEKAAWLQAQEQCQAWAGAALAMVDSPAVQRFLVSRVTRSLDVWIGFSTVQGVEVGPAPQGEAFSLESCQNWLPGEPHPATAEHCVRLGPTGWCNTDLCSAPHSYVCELQPGGPVQDAENLLVGAPSGDLQGPLTPLAQQDGLSAPHEPVEVMVFPGLRLSREAFLTTAEFGTQELRRPAQLRLQVYRLLSTAGTPENGSEPESRSPDNRTQLAPACMPGGRWCPGANICLPLDASCHPQACANGCTSGPGLPGAPYALWREFLFSVPAGPPAQYSVTLHGQDVLMLPGDLVGLQHDAGPGALLHCSPAPGHPGPQAPYLSANASSWLPHLPAQLEGTWACPACALRLLAATEQLTVLLGLRPNPGLRLPGRYEVRAEVGNGVSRHNLSCSFDVVSPVAGLRVIYPAPRDGRLYVPTNGSALVLQVDSGANATATARWPGGSVSARFENVCPALVATFVPGCPWETNDTLFSVVALPWLSEGEHVVDVVVENSASRANLSLRVTAEEPICGLRATPSPEARVLQGVLVRYSPVVEAGSDMVFRWTINDKQSLTFQNVVFNVIYQSAAVFKLSLTASNHVSNVTVNYNVTVERMNRMQGLQVSTVPAVLSPNATLALTAGVLVDSAVEVAFLWTFGDGEQALHQFQPPYNESFPVPDPSVAQVLVEHNVMHTYAAPGEYLLTVLASNAFENLTQQVPVSVRASLPSVAVGVSDGVLVAGRPVTFYPHPLPSPGGVLYTWDFGDGSPVLTQSQPAANHTYASRGTYHVRLEVNNTVSGAAAQADVRVFEELRGLSVDMSLAVEQGAPVVVSAAVQTGDNITWTFDMGDGTVLSGPEATVEHVYLRAQNCTVTVGAASPAGHLARSLHVLVFVLEVLRVEPAACIPTQPDARLTAYVTGNPAHYLFDWTFGDGSSNTTVRGCPTVTHNFTRSGTFPLALVLSSRVNRAHYFTSICVEPEVGNVTLQPERQFVQLGDEAWLVACAWPPFPYRYTWDFGTEEAAPTRARGPEVTFIYRDPGSYLVTVTASNNISAANDSALVEVQEPVLVTSIKVNGSLGLELQQPYLFSAVGRGRPASYLWDLGDGGWLEGPEVTHAYNSTGDFTVRVAGWNEVSRSEAWLNVTVKRRVRGLVVNASRTVVPLNGSVSFSTSLEAGSDVRYSWVLCDRCTPIPGGPTISYTFRSVGTFNIIVTAENEVGSAQDSIFVYVLQLIEGLQVVGGGRYFPTNHTVQLQAVVRDGTNVSYSWTAWRDRGPALAGSGKGFSLTVLEAGTYHVQLRATNMLGSAWADCTMDFVEPVGWLMVAASPNPAAVNTSVTLSAELAGGSGVVYTWSLEEGLSWETSEPFTTHSFPTPGLHLVTMTAGNPLGSANATVEVDVQVPVSGLSIRASEPGHHHHHHHHHH

SEQ ID NO: 16 - DNA, 2358 bp, Artificial Sequence
  Synthetic Polynucleotide
aaccgctcggcgcagtttgaggccgccaccagccccagcccccggcgtgtggcctaccactgggactttggggatgggtcgccagggcaggacacagatgagcccagggccgagcactcctacctgaggcctggggactaccgcgtgcaggtgaacgcctccaacctggtgagcttcttcgtggcgcaggccacggtgaccgtccaggtgctggcctgccgggagccggaggtggacgtggtcctgcccctgcaggtgctgatgcggcgatcacagcgcaactacttggaggcccacgttgacctgcgcgactgcgtcacctaccagactgagtaccgctgggaggtgtatcgcaccgccagctgccagcggccggggcgcccagcgcgtgtggccctgcccggcgtggacgtgagccggcctcggctggtgctgccgcggctggcgctgcctgtggggcactactgctttgtgtttgtcgtgtcatttggggacacgccactgacacagagcatccaggccaatgtgacggtggcccccgagcgcctggtgcccatcattgagggtggctcataccgcgtgtggtcagacacacgggacctggtgctggatgggagcgagtcctacgaccccaacctggaggacggcgaccagacgccgctcagtttccactgggcctgtgtggcttcgacacagagggaggctggcgggtgtgcgctgaactttgggccccgcgggagcagcacggtcaccattccacgggagcggctggcggctggcgtggagtacaccttcagcctgaccgtgtggaaggccggccgcaaggaggaggccaccaaccagacggtgctgatccggagtggccgggtgcccattgtgtccttggagtgtgtgtcctgcaaggcacaggccgtgtacgaagtgagccgcagctcctacgtgtacttggagggccgctgcctcaattgcagcagcggctccaagcgagggcggtgggctgcacgtacgttcagcaacaagacgctggtgctggatgagaccaccacatccacgggcagtgcaggcatgcgactggtgctgcggcggggcgtgctgcgggacggcgagggatacaccttcacgctcacggtgctgggccgctctggcgaggaggagggctgcgcctccatccgcctgtcccccaaccgcccgccgctggggggctcttgccgcctcttcccactgggcgctgtgcacgccctcaccaccaaggtgcacttcgaatgcacgggctggcatgacgcggaggatgctggcgccccgctggtgtacgccctgctgctgcggcgctgtcgccagggccactgcgaggagttctgtgtctacaagggcagcctctccagctacggagccgtgctgcccccgggtttcaggccacacttcgaggtgggcctggccgtggtggtgcaggaccagctgggagccgctgtggtcgccctcaacaggtctttggccatcaccctcccagagcccaacggcagcgcaacggggctcacagtctggctgcacgggctcaccgctagtgtgctcccagggctgctgcggcaggccgatccccagcacgtcatcgagtactcgttggccctggtcaccgtgctgaacgagtacgagcgggccctggacgtggcggcagagcccaagcacgagcggcagcaccgagcccagatacgcaagaacatcacggagactctggtgtccctgagggtccacactgtggatgacatccagcagatcgctgctgcgctggcccagtgcatggggcccagcagggagctcgtatgccgctcgtgcctgaagcagacgctgcacaagctggaggccatgatgctcatcctgcaggcagagaccaccgcgggcaccgtgacgcccaccgccatcggagacagcatcctcaacatcacaggagacctcatccacctggccagctcggacgtgcgggcaccacagccctcagagctgggagccgagtcaccatctcggatggtggcgtcccaggcctacaacctgacctctgccctcatgcgcatcctcatgcgctcccgcgtgctcaacgaggagcccctgacgctggcgggcgaggagatcgtggcccagggcaagcgctcggacccgcggagcctgctgtgctatggcggcgccccagggcctggctgccacttctccatccccgaggctttcagcggggccctggccaacctcagtgacgtggtgcagctcatctttctggtggactccaatccctttccctttggctatatcagcaactacaccgtctccaccaaggtgcaccatcaccatcaccatcaccatcaccattag

SEQ ID NO: 17 - protein, 785 aa, Artificial Sequence
  Synthetic Polypeptide
NRSAQFEAATSPSPRRVAYHWDFGDGSPGQDTDEPRAEHSYLRPGDYRVQVNASNLVSFFVAQATVTVQVLACREPEVDVVLPLQVLMRRSQRNYLEAHVDLRDCVTYQTEYRWEVYRTASCQRPGRPARVALPGVDVSRPRLVLPRLALPVGHYCFVFVVSFGDTPLTQSIQANVTVAPERLVPIIEGGSYRVWSDTRDLVLDGSESYDPNLEDGDQTPLSFHWACVASTQREAGGCALNFGPRGSSTVTIPRERLAAGVEYTFSLTVWKAGRKEEATNQTVLIRSGRVPIVSLECVSCKAQAVYEVSRSSYVYLEGRCLNCSSGSKRGRWAARTFSNKTLVLDETTTSTGSAGMRLVLRRGVLRDGEGYTFTLTVLGRSGEEEGCASIRLSPNRPPLGGSCRLFPLGAVHALTTKVHFECTGWHDAEDAGAPLVYALLLRRCRQGHCEEFCVYKGSLSSYGAVLPPGFRPHFEVGLAVVVQDQLGAAVVALNRSLAITLPEPNGSATGLTVWLHGLTASVLPGLLRQADPQHVIEYSLALVTVLNEYERALDVAAEPKHERQHRAQIRKNITETLVSLRVHTVDDIQQIAAALAQCMGPSRELVCRSCLKQTLHKLEAMMLILQAETTAGTVTPTAIGDSILNITGDLIHLASSDVRAPQPSELGAESPSRMVASQAYNLTSALMRILMRSRVLNEEPLTLAGEEIVAQGKRSDPRSLLCYGGAPGPGCHFSIPEAFSGALANLSDVVQLIFLVDSNPFPFGYISNYTVSTKVHHHHHHHHHH

SEQ ID NO: 18 - DNA, 1821 bp, Artificial Sequence
  Synthetic Polynucleotide
atggccgaggaggcagcccagaacatttctgatgaccaggaaaggtgtctccaggctgcctgctgcctttcctttggtggtgagctgtctgtgagcactgacaagagctggggtcttcatctgtgcagctgtagccctcctggaggtggattgtgggtcgaggtctatgctaatcatgtgcttcttatgagtgatgggaagtgtggctgtccttggtgtgctctgaatggaaaggcagaagaccgggaatcacagagcccatcctcatcagcttccaggcagaagaacatttggaaaacaactagcgaagcagcgttaagtgttgttaatgaaaaaacacaggctgttgttaatgaaaaaacacaggcgcctctggattgtgataacagtgctgatagaattccccacaaacctttcattataatcgcaagggcctggagcagtggtggccccaggttccatcacaggcggctgtgtgctactgggaccgcagacagcacattctctgctcttctccagctccagggcaccacttctgcagcagctccctgcagcctgaagatggaggcttcctgctgtgtcctgagactgctgtgctgtgcggaggatgtggccacggggctgcttcctgggactgtcacgatggagacccccaccaaggtggccagacccactcagaccagctcccagcgagtgcccctgtggccgatttcacattttcccacttctcccagaagctcccacggccttccgcctggcattcctcgcacccccagcttcacggcatcgcagtctggttctgagatcctctatccccctactcagcatcctcctgtggccatcctagctcgaaattctgataacttcatgaaccctgttcttaattgctccctggaagtggaagctcgggcacctccaaatctgggattccgtgttcatatggcttctggagaggctctctgtctgatgatggatttcggggacagttctggggttgaaatgaggctacacaacatgtctgaggcaatggcggtgactgcctaccaccagtactcaaaaggtattttttttcatcttttacattttcagttggatatgtccacctacaaagaagcagagacacaaaatacaactttaaatgtttacttgtgccaaagtgaaaacagctgcctggaagactcagaccccagtaaccttggatatgagcttatttctgcctttgtcaccaaaggagtctatatgctcaaggctgttatttataacgagtttcatggaaccgaagtggagcttgggccttattatgtggagattggccatgaggccgtgtctgcgttcatgaactccagcagtgtccatgaagatgaagtgcttgtctttgctgactcccaagtgaatcagaaaagcactgtggttatacatcactttccatctattccttcatataacgtgtcctttatttctcagactcaagtgggtgacagccaggcttggcacagcatgactgtctggtataagatgcaatccgtctctgtctacacaaatggaactgtgtttgccacagacacagacattacatttacagctgttaccaaggaaacaatacccctggaatttgagtggtattttggagaggacccaccagtgaggacaacttcaagaagcattaaaaaaagactcagcatcccccaatggtatcgtgtgatggttaaggcttccaacaggatgagcagtgtggtctctgagccccatgtcatcagggtgcagaagaaaattgtggccaatcggctccaccatcaccatcaccatcaccatcaccattag

SEQ ID NO: 19 - protein, 606 aa, Artificial Sequence
  Synthetic Polypeptide
MAEEAAQNISDDQERCLQAACCLSFGGELSVSTDKSWGLHLCSCSPPGGGLWVEVYANHVLLMSDGKCGCPWCALNGKAEDRESQSPSSSASRQKNIWKTTSEAALSVVNEKTQAVVNEKTQAPLDCDNSADRIPHKPFIIIARAWSSGGPRFHHRRLCATGTADSTFSALLQLQGTTSAAAPCSLKMEASCCVLRLLCCAEDVATGLLPGTVTMETPTKVARPTQTSSQRVPLWPISHFPTSPRSSHGLPPGIPRTPSFTASQSGSEILYPPTQHPPVAILARNSDNFMNPVLNCSLEVEARAPPNLGFRVHMASGEALCLMMDFGDSSGVEMRLHNMSEAMAVTAYHQYSKGIFFHLLHFQLDMSTYKEAETQNTTLNVYLCQSENSCLEDSDPSNLGYELISAFVTKGVYMLKAVIYNEFHGTEVELGPYYVEIGHEAVSAFMNSSSVHEDEVLVFADSQVNQKSTVVIHHFPSIPSYNVSFISQTQVGDSQAWHSMTVWYKMQSVSVYTNGTVFATDTDITFTAVTKETIPLEFEWYFGEDPPVRTTSRSIKKRLSIPQWYRVMVKASNRMSSVVSEPHVIRVQKKIVANRLHHHHHHHHHH

SEQ ID NO: 20 - protein, 9 aa, Artificial Sequence
  Synthetic Polypeptide
YPYDVPDYA

SEQ ID NO: 21 - protein, 24 aa, Artificial Sequence
  Synthetic Polypeptide
QAVDNLTSAPGNTSLCTRDYKITQ

SEQ ID NO: 22 - protein, 4 aa, Artificial Sequence
  Synthetic Polypeptide
MVWR

SEQ ID NO: 23 - protein, 9 aa, Artificial Sequence
  Synthetic Polypeptide
TEGLLPLEE

SEQ ID NO: 24 - protein, 12 aa, Artificial Sequence
  Synthetic Polypeptide
TSPRQAVCLTRM

SEQ ID NO: 25 - protein, 14 aa, Artificial Sequence
  Synthetic Polypeptide
LTAFGASLFVPPSH

SEQ ID NO: 26 - protein, 4 aa, Artificial Sequence
  Synthetic Polypeptide
REWK

SEQ ID NO: 27 - protein, 9 aa, Artificial Sequence
  Synthetic Polypeptide
SERFSPQPG

SEQ ID NO: 28 - protein, 12 aa, Artificial Sequence
  Synthetic Polypeptide
TSPEKVNCSYHR

SEQ ID NO: 29 - protein, 14 aa, Artificial Sequence
  Synthetic Polypeptide
LAAFALLRRKLKAS

SEQ ID NO: 30 - protein, 5 aa, Artificial Sequence
  Synthetic Polypeptide
QETWD

SEQ ID NO: 31 - protein, 9 aa, Artificial Sequence
  Synthetic Polypeptide
DSGCQVGPR

SEQ ID NO: 32 - protein, 12 aa, Artificial Sequence
  Synthetic Polypeptide
TSPYQTHCLCNM

SEQ ID NO: 33 - protein, 14 aa, Artificial Sequence
  Synthetic Polypeptide
LTFFGSTFLVMPNA

SEQ ID NO: 34 - protein, 5 aa, Artificial Sequence
  Synthetic Polypeptide
NQTWS

SEQ ID NO: 35 - protein, 9 aa, Artificial Sequence
  Synthetic Polypeptide
SAGCQVGPQ

SEQ ID NO: 36 - protein, 12 aa, Artificial Sequence
  Synthetic Polypeptide
STILRTQCLCNM

SEQ ID NO: 37 - protein, 14 aa, Artificial Sequence
  Synthetic Polypeptide
LTFFASDFFVVPRT

SEQ ID NO: 38 - protein, 5 aa, Artificial Sequence
  Synthetic Polypeptide
QSEWR

SEQ ID NO: 39 - protein, 9 aa, Artificial Sequence
  Synthetic Polypeptide
EGYCILGEK

SEQ ID NO: 40 - protein, 12 aa, Artificial Sequence
  Synthetic Polypeptide
TSWYEVHCICKN

SEQ ID NO: 41 - protein, 14 aa, Artificial Sequence
  Synthetic Polypeptide
VVRARRQLGTIGLT

SEQ ID NO: 42 - protein, 8 aa, Artificial Sequence
  Synthetic Polypeptide
DYKDDDDK

SEQ ID NO: 43 - protein, 120 aa, Homo sapiens
HFRWSALQVSVGLYTSLCQYFSEEDMVWRTEGLLPLEETSPRQAVCLTRHLTAFGASLFVPPSHVRFVFPEPTADVNYIVMLTCAVCLVTYMVMAAILHKLDQLDASRGRAIPFCGQRGR

SEQ ID NO: 44 - protein, 120 aa, Mus musculus
HFHWSALEVSVGLYTSLCQYFSEEMMMWRTEGIVPLEETSPSQAVCLTRHLTAFGASLFVPPSHVQFIFPEPSASINYIVLLTCVICLVTYVVMAMILRKLDQLDVSRVRVIPFCGKGGR

SEQ ID NO: 45 - protein, 120 aa, Rattus norvegicus
HFHWSALEVSVGLYTSLCQYFSEEAMMWRTEGIVPLEETSPSQAVCLTRHLTAFGASLFVPPSHVQFIFPEPSVSINYIVLLTCVICLVTYVIMAMILRKLDQLDVSRVRVIPFCGKGGR

SEQ ID NO: 46 - protein, 120 aa, Homo sapiens
RYLAKAVNYTVHFQWIRCLFWDKREWKSERFSPQPGTSPEKVNCSYHRLAAFALLRRKLKASFEVSDISKLQSHPENLLPSIFIMGSVILYGFLVAKSRQVDHHEKKKAGYIFLQEASLP

SEQ ID NO: 47 - protein, 120 aa, Mus musculus
KYLAGAVNYTVHFQWIQCVFWDKTEWRSEGPYPQPGSSPEKVNCSYHHLAPVSVLRRKLNATLEVSSISEFQSHPHNLLPGIFSAFLLVLYGILVSKSRYVDCHEKKNPGFIFLEEDTLP

SEQ ID NO: 48 - protein, 120 aa, Rattus norvegicus
KYLAGAVNYTVHFQWIQCVFWDKTEWRSEGPCPQPGTSPEKVNCSYHRLAPFSVLRRKLNATFQVSSVSEFQSHPHNLLPSIFSVFFLVLYGFLMSKSSCVDCHKKKDPGCIFLEEETLL